(19) United States
(12) Patent Application Publication
(10) Pub. No.: US 2006/0272652 A1
(43) Pub. Date: Dec. 7, 2006

(54) Virtual patient software system for educating and treating individuals with diabetes

(75) Inventors: David N. Stocker, Santa Monica, CA (US); Sami Kanderian JR., Northridge, CA (US); Gabriel J. Cortina, Camarillo, CA (US); Tamir Nitzan, Canyon Country, CA (US); Jon Plummer, Los Angeles, CA (US); Garry M. Steil, Pasadena, CA (US); John J. Mastrototaro, Los Angeles, CA (US)

Correspondence Address: PILLSBURY WINTHROP SHAW PITTMAN LLP, P.O BOX 10500, McLean, VA, 22102, US

(73) Assignee: MEDTRONIC MINIMED, INC., Northridge, CA (US)

(21) Appl. No.: 11/145,485

(22) Filed: Jun. 3, 2005

(51) Int. Cl. A61B 19/00

(52) U.S. Cl. 128/898

(57) ABSTRACT

A system to assist an individual in developing a therapy in diabetes treatment of a patient includes a user interface control module, a simulation engine, a charting and display module. The user interface control module receives an input related to the patient and captures a current time of the simulation. The simulation engine receives the input, generates a plurality of blood glucose readings for the patient up to the current time of the simulation based on the input, and to transfers the plurality of blood glucose readings. The charting and display module receives the plurality of blood glucose readings and display the plurality of blood glucose readings. The simulation engine receives patient parameters from a patient parameter library based on a selected patient model.

### BACKGROUND

1. Technical Field

Embodiments of this invention relate generally to a method and apparatus for assisting patients and doctors in managing insulin delivery to diabetes. Specifically, the invention relates to a virtual patient software system that provides a patient and/or a medical professional to monitor blood glucose levels in response to the modification of different aspects of insulin delivery, food intake, and an exercise program.

2. Discussion of the Related Art

A blood glucose metabolism is controlled by a myriad of processes, and is optimized to achieve normoglycemia even for wide swings in predominant effect inputs, food, and physical exercise. Illness is characterized by the inability to control over one or more biological processes. Disease management is one solution for people suffering from incurable afflictions. In other words, careful monitoring of parameters of interest, couple with corrective actions such as insulin injections (multiple daily injections or continuous subcutaneous insulin infusion through an insulin pump) result in effective disease management.

For example, diabetes patients have little or no endogenous blood glucose (BG) control capability and may must inject insulin or infuse insulin to compensate for swings outside the acceptable serum glucose range. Diabetes mellitus is a significant disease and its incidence rate has been increasing during the last twenty to thirty years. Studies have indicated that tight control of blood glucose levels results in a dramatic reduction of complications. Tight control of blood glucose levels requires that patients with this condition intensively monitor blood sugar measurements, take insulin shots more frequently to address blood glucose irregularities, and/or infuse insulin via an insulin pump on a regular basis to maintain an acceptable blood glucose level.

Improved technology has allowed patients to infuse insulin or inject insulin at home as well as monitor glucose levels at home. Monitoring of blood glucose levels includes the utilization of home-use blood glucose meter systems, where a user pricks a finger to draw blood, places the blood on a fingerstrip, which is used in conduction with the blood glucose meter toe measure the blood glucose level. These type of measurements provide a user with blood glucose readings at specified moments in time, but do not provide an accurate indication of continuous blood glucose levels. Illustratively, on a graph that displayed blood glucose levels over time, if fingerstrip measurements are utilized, the graph would only display datapoints corresponding to the user's readings and would not show swings of the blood glucose level in between the times the finger sticks were taken. This results in a patient or even a medical professional not being able to completely accurately predict the shape of the blood glucose level curve in between datapoints.

Patients can also utilize a sub-cutaneous glucose sensor that is inserted in part of a patient's skin (such as around the hips or right above the hip area or near the stomach area), which measures blood glucose levels within a patient. The glucose sensor is able to monitor blood glucose levels on a periodic basis. Under certain operating conditions, the glucose sensor is able to monitor blood glucose levels on a continuous basis. This results in a more accurate picture of the patient's blood glucose level because more readings are taking place.

While these tools provide a good understanding of a patient's blood glucose level during specific periods of times (over certain times), there is no teaching or educational tool that quickly (in real-time) provides a patient or a medical professional (i.e., doctor, nurse practitioner, or patient) with simulated information regarding the effects of certain intakes or treatments on a patient's blood glucose level. In addition, there is no tool that quickly or in real-time provides a patient with personalized information regarding the effects of certain intakes or treatments on the actual patient's blood glucose levels. An AIDA Interactive Diabetes Advisor software allows a user of the system, over the intranet, to enter in the user's predicted meals, exercise schedule, and anticipated intake of insulin (via boluses, shots, and or insulin pumps) for a specified time period (such as 24 hours). The AIDA software predicts the blood glucose level based on the input supplied by the user. This software may be extremely helpful for patients who have a very regimented schedule that can always be followed, but would not produce accurate results in real-time environments. The AIDA software is not designed for real-time or almost real-time interaction.

Accordingly, a need exists to provide both diabetes patients and medical professionals with an interactive visual teaching tool that illustrates the effects of certain intakes and events on blood glucose levels and presents this information in an easy-to-read and understandable user format.

### BRIEF DESCRIPTION OF THE DRAWINGS

FIG. 1(a) illustrates a block diagram and dataflow diagram of a computing device incorporation a virtual patient software program according to an embodiment of the present invention;

FIG. 1(b) illustrates a virtual patient software system utilizing real or actual patient data according to an embodiment of the present invention;

FIG. 2 illustrates an initial screen of the Virtual Patient software system according to an embodiment of the invention;

FIG. 3(a) illustrates a bolus input window and a bolus wizard window according to an embodiment of the present invention;

FIG. 3(b) illustrates an exercise input screen in a patient manipulate and view menu according to an embodiment of the present invention;

FIG. 3(c) illustrates a basal adjustment rate menu in a patient interactive manipulate and view screen according to an embodiment of the invention;

FIG. 3(d) illustrates a carbohydrate determination menu according to an embodiment of the present invention;

FIG. 4(a) illustrates a flowchart for a patient mode Virtual Patient software according to an embodiment of the present invention;

FIG. 4(b) illustrates a flowchart of a doctor interaction model of the virtual patient software according to an embodiment of the present invention;

FIG. 5 illustrates the interaction selection screen of the Virtual Patient software;

FIG. 6 illustrates a patient selection screen of the Virtual Patient software according to an embodiment of the present invention;

FIG. 7 illustrates a patient manipulate and view screen according to an embodiment of the present invention;

FIG. 8 illustrates an manipulate and view screen 700 of the virtual patient software according to an embodiment of the present invention;

FIG. 9 illustrates a graph display section of a manipulate and view screen of the virtual patient software according to an embodiment of the present invention;

FIG. 10 illustrates a presenting screen 1000 for a patient, e.g., Megan, in the doctor interaction mode of the virtual patient software according to an embodiment of the present invention;

FIG. 11 illustrates a doctor interaction manipulate and view screen according to an embodiment of the present invention;

FIG. 12 displays a doctor interaction menu including a bolus input screen according to an embodiment of the present invention;

FIG. 12(b) illustrates a doctor manipulate and view screen where the adjust carb/insulin ration has been selected;

FIG. 13 illustrates a lab report displayed in a patient interaction screen according to an embodiment of the present invention;

FIG. 14(a) displays a doctor interaction manipulate and view screen being displayed in an around meals view according to an embodiment of the invention;

FIG. 14(b) displays a doctor manipulate and view screen being displayed in a modal view according to an embodiment of the present invention;

FIG. 14(c) illustrates a longitudinal view of the doctor view and manipulate menu according to an embodiment of the invention;

FIG. 15 illustrates a closed-loop system including a glucose sensor, a computing device having virtual patient software, and an insulin pump according to an embodiment of the present invention;

FIG. 16 illustrates a flowchart of operation for customized virtual patient software according to an embodiment of the present invention;

FIGS. 17(a)-17(h) illustrate a sample use of the virtual patient software in a patient mode according to an embodiment of the present invention; and

FIGS. 18(a)-18(e) illustrate a sample use of the virtual patient software in a doctor mode according to an embodiment of the present invention.

### DETAILED DESCRIPTION OF EMBODIMENTS OF THE INVENTION

In an embodiment of the invention, the virtual patient software make be utilized in an educational fashion utilizing models of virtual patients. A patient or a doctor may utilize the virtual patient software in the educational fashion. In an embodiment of the invention, the virtual patient software may morph into more of an actual patient management tool because the patient model will have been developed specifically for the patient and a patient's insulin pump or insulin sensor may provide readings and information to the virtual patient software.

The present invention described below with reference to flowchart illustrations of methods, apparatus, and computer program products. It will be understood that each block of the flowchart illustrations, and combinations of blocks in the flowchart illustrations, can be implemented by computer program instructions (as can any menu screens described in the Figures). These computer program instructions may be loaded onto a computer or other programmable data processing apparatus to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks, and/or menus presented herein.

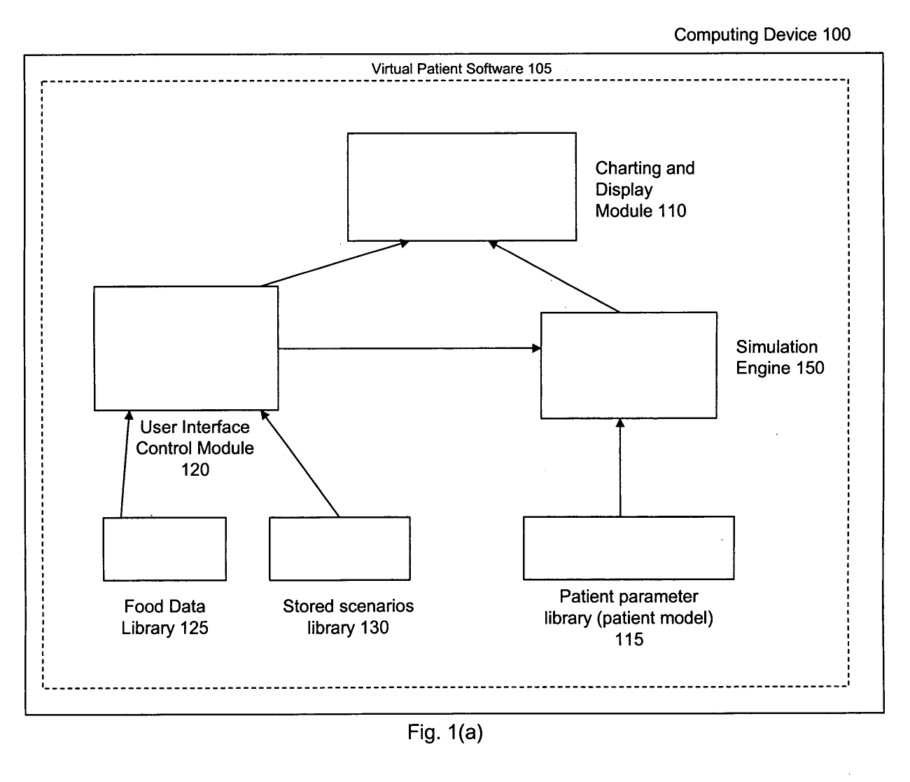

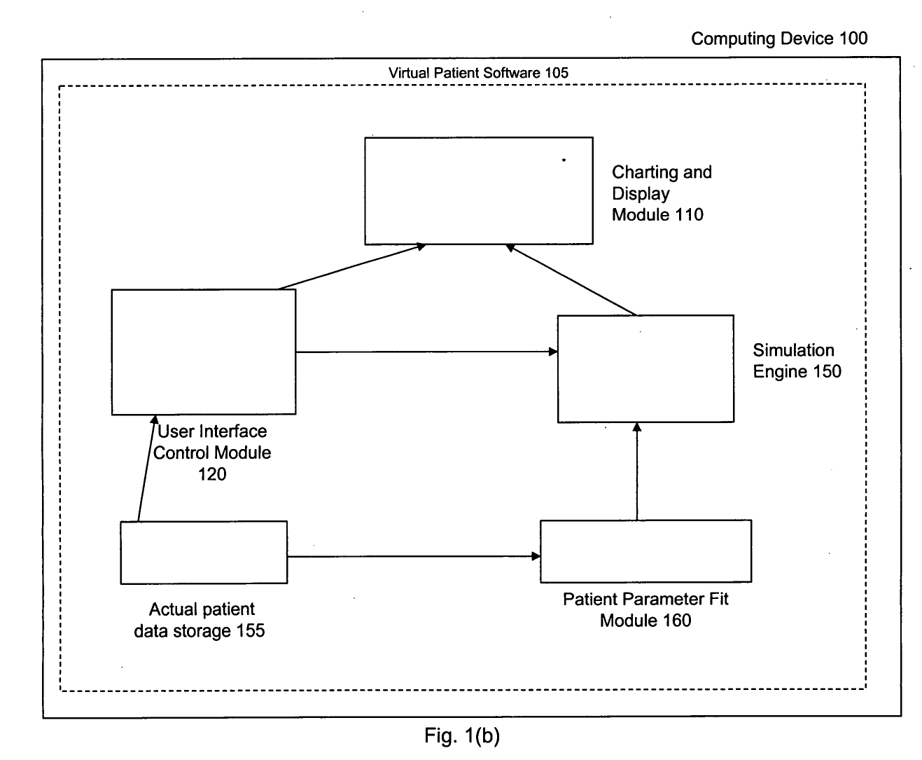

FIG. 1(a) illustrates a block diagram and dataflow diagram of a computing device incorporation a virtual patient software program according to an embodiment of the present invention. A computing device 100 includes the Virtual Patient software 105. In the embodiment of the invention illustrated in FIG. 1(a), the virtual patient software 105 includes a charting and display module 110, a user interface control module 120, a food data library 125, a patient parameter library 115, and a simulation engine 150. In a embodiment of the invention, the virtual patient software 105 may include a stored scenarios library 130.

The computing device 100 hosting the Virtual Patient software 105 may be, but is not limited to, a desktop computer, a laptop computer, a server, a network computer, a personal digital assistant (PDA), a portable telephone including computer functions, an insulin pump including a display, a glucose sensor including a display, a glucose meter including a display, and/or a combination insulin pump/glucose sensor. The computing device 100 may also be a server located on the Internet that is accessible via a browser installed on a laptop computer, desktop computer, a network computer, or a PDA.

In an embodiment of the invention, the virtual patient software 105 may be installed on a computing device 100 where the computing device 100 includes the Microsoft.NET™ framework. In other embodiments of the invention, the virtual patient software 105 may be written in the Java programming language and installed on a Java-enabled machine.

When the Virtual Patient software 105 is initialized on the computing device 100, the user interface control module 120 displays an input screen allowing a user to select a mode of operation. Illustratively, modes of operation may include a patient simulation mode or a medical professional (e.g., doctor or nurse practitioner) simulation mode. In alternative embodiments of the invention, the modes of operation may include a patient interaction mode or a medical professional interaction mode. In an embodiment of the invention, the user interface control module 120 may access or store different web pages or active server pages for the different modes of operation. For example, the user interface control module 120 may include a number of web pages, active server pages, or screen shots for the patient simulation mode or may be able to access the web pages, active server pages, or screen shots. The user interface control module 120 may also include information to address how the web pages, active server pages, or screen shots interact with each other.

In an embodiment of the invention, if the doctor's mode is selected, e.g., the medical professional mode, the user interface and control module 120 may access a stored scenarios library 130 to extract the scenarios applicable to the selected patient model. Under certain operating conditions, the information supplied from the stored scenarios library 130 is transferred to the user interface control module 120. Under other operating conditions, the user interface control module 110 may access the stored scenarios library 130 in response to the entering of inputs, events, or activities. If the patient mode of the virtual patient software 105 is selected, the user interface control module 120 may also utilize the stored scenarios library 130 to provide the virtual patient software with the data for the different scenarios that are available to be selected by the user, as discussed below.

After the mode is selected, the user interface control module 120 presents a plurality of patient metabolic models (which may be referred to as patient models). The patient models is utilized to either simulate a patient's reaction to different events and activities or to provide an actual reaction to different events and activities. The patient parameter library 115 stores different parameters for different patient models. For example, if six patients are able to be selected, then six sets of patient parameters may be stored in the patient parameter library 115. Having a plurality of sets of patient parameters stored in the patient parameter library 115 provides a user with the ability to select from a number of metabolic models, e.g., such as a model corresponding to a young child, a woman with gestational diabetes, or a middle-age man with adult-onset diabetes.

In embodiments of the invention, the patient parameters may be for actual patients or for virtual patients, e.g., patients with certain characteristics. Many metabolic models for use in diabetes treatment have been developed and are known in art. In an embodiment of the invention, a new metabolic model, as described below, may utilize patient parameters from the patient parameter library, and the new metabolic model may provide the mathematical algorithms to the simulation engine in order to have the mathematical algorithms run by the simulation engine 150.

The Medtronic MiniMed model is based on a minimal set of assumptions. An assumed or measured insulin profile, in response to single bolus of insulin, is taken as an impulse response I(t). From this response, plasma insulin concentration (Ip(t)), for arbitrary insulin delivery, such at might occur with an insulin pump, is described by the convolution of the impulse response (I(t)) and the arbitrary insulin delivery (ID(t)) profile:

$${I}_{p} ( t ) = {\int }_{0}^{t} I ( t - τ ) ID ( τ ) ⅆ τ \qquad Equation 1$$

In the preferred embodiment, I(t) is described by a biexponential curve characterized by two time delays (1/α₁ and 1/α₂ in units of minutes) and a scaling factor “A” (units of insulin concentration; typically μU/ml or pmol/L):
I(t)=A·[e^{-α}^{₁}^{t}−e^{-α}^{₂}^{t}] Equation 2
For ease of implementation, equations 1 and 2 can be expressed as a series of differential equations:

$$\frac{ⅆ {I}_{r}}{ⅆ t} = - {α}_{1} {I}_{r} + A \cdot ID ( t ) \\ \frac{ⅆ {I}_{p}}{ⅆ t} = - {α}_{2} {I}_{p} + ( {α}_{2} - {α}_{1} ) \cdot {I}_{r} ( t ) \qquad Equation 3$$

where the variable Iᵣ(t) reflects insulin in a remote compartment.

Insulin effects a change in glucose concentration by increasing peripheral glucose uptake and decreasing endogenous glucose production. Glucose uptake is assumed to be proportional to glucose concentration; endogenous glucose production is assumed inversely proportional to glucose concentration. In both instances the effect of insulin is to increase the proportional rate. The overall effect does not occur simultaneously with changes in plasma insulin concentration. In the preferred embodiment, the delay time for the two effects is assumed to be the same, and the time complete time profile (X(t)) is described by a first order differential equation:

$$\frac{ⅆ X ( t )}{ⅆ t} = - {p}_{2} X ( t ) + {p}_{3} ( {I}_{p} ( t ) - {I}_{B} ) \qquad Equation 4$$

In this form, Iₚ(t) is the plasma insulin concentration (Equation 2), I_{B} is the basal insulin concentration required to maintain glucose concentration at a desired basal level (G_{B}), 1/p₂ defines the time constant for insulin action (minutes), and p3/p2 defines the subject's insulin sensitivity (typical units min⁻¹ per μU/ml).

Glucose concentration increases in response to exogenous glucose appearance (meals); however, following a meal, glucose appearance into the plasma space does not occur instantly. The time course can be very complex with multiple factors affecting the rate of appearance (e.g. percent fat content). Several models have been proposed to describe this curve (e.g., AIDA). The preferred embodiment used in the Medtronic MiniMed is to describe the total number of carbohydrates (CHO) during a meal as appearing as an impulse (δ(t)), with subsequent rate of appearance into a remote space (R_{aR}), and then appearance into the plasma space with rate R_{aP}(t):

$$\frac{ⅆ {R}_{aR}}{ⅆ t} = - {p}_{4} \cdot {R}_{aR} + {p}_{4} \cdot CHO \cdot δ ( t ) \\ \frac{ⅆ {R}_{aP}}{ⅆ t} = - {p}_{5} \cdot {R}_{aP} + {p}_{5} \cdot {R}_{aR} \qquad Equation 5$$

In this formulation, p₄ characterizes the rate of carbohydrate appearance into a remote compartment, and p₅ characterizes the rate of transfer from the remote compartment into plasma (p₄ can equal p₅).

Each of the individual effects and processes (equations 3 through 5) contribute to changes in glucose concentration (G(t)). The final, or preferred embodiment describing these changes is:

$$\frac{ⅆ G}{ⅆ t} = - ( {p}_{1} + X ) G + {p}_{1} {G}_{B} \frac{1}{V} {R}_{aP} ( t ) \\ \frac{ⅆ X}{ⅆ t} = - {p}_{2} X + {p}_{3} ( {I}_{p} ( t ) - {I}_{B} ) \\ \frac{ⅆ {I}_{r}}{ⅆ t} = - {α}_{1} {I}_{r} + A \cdot ID ( t ) \\ \frac{ⅆ {I}_{p}}{ⅆ t} = - {α}_{2} {I}_{p} + ( {α}_{2} - {α}_{1} ) \cdot {I}_{r} ( t ) \\ \frac{ⅆ {R}_{aR}}{ⅆ t} = - {p}_{4} \cdot {R}_{aR} + {p}_{4} \cdot CHO \cdot δ ( t ) \\ \frac{ⅆ {R}_{aP}}{ⅆ t} = - {p}_{5} \cdot {R}_{aP} + {p}_{5} \cdot {R}_{aR} \qquad Equation 6$$

In this embodiment (equation 6), glucose concentration (G(t); typical units of mg/dl) increases in response to the rate of glucose appearance into plasma (Rₐₚ(t); typical units equal mg/min) with the effect size dependent on the glucose distribution volume (V, units of dl; typical estimate equal 65% of extracellular space). Insulin decreases glucose concentration in proportion to the prevailing glucose concentration. Parameter p₁ reflects the effect of glucose per se to increase glucose disposal and decrease endogenous glucose production; p₁G_{B} reflects the endogenous glucose appearance (mg/min per dl) normalized to the glucose distribution volume measured under a steady state plasma insulin concentration (I_{B}; typically measured under basal or steady-state fasting conditions).

Enhanced versions of the model include the ability to make all parameters (p1-p5, α₁,α₂, A) time dependant, add separate descriptions of multiple glucose compartments, introduce nonlinearities into any or all processes, and interrelate various meal (p4,p5) and metabolic (p1-p3) parameters.

After an interactive mode (patient or doctor) has been selected and the patient model has been selected, the user interface control module 120 may allow for inputs, activities, or events to be entered into the virtual patient software 105. For example, if the patient mode has been selected and the child (Kevin) metabolic model has been selected because of the patient's similar characteristics to Kevin's model, a user of the virtual patient software 105 may enter a number of bolus units that has been taken, carbohydrates that have been eaten, or whether or not a basal rate of insulin from an insulin pump has been adjusted. In addition, a user of the virtual patient software may also enter when and how long the patient has exercised, etc. In addition, a user of the virtual patient software 105 may identify that a sensor reading was taken or that a fingerstick was taken and a blood glucose meter provided a reading.

The user interface control module 120 receives the entered inputs, activities, or events. In an embodiment of the invention, the user interface control module 120 may transfer the entered inputs, activities, or events to the charting and display module 110 for presentation on a display of the computing device 100. In an embodiment of the invention, the charting and display module 110 may present a single graph on the display of the computing device 100. Under other operating conditions, the charting and display module 110 may present a plurality of graphs on the display of the computing device 100. For example, the charting and display module 110 may display information on how many carbohydrates were consumed, what insulin has been ingested, and how much the patient has exercised.

After an input, activity, or events is entered, the user interface control module 120 transfers the entered information to a simulation engine 150. Under certain operating conditions, the patient parameter library 115 supplies the patient's parameters (which were selected based on a user's selection of a patient model earlier) to the simulation engine 150. Under certain operating conditions, the patient parameter library 115 may transfer baseline data to the simulation engine 150. In this manner, the simulation engine 150 may have baseline data to be utilized to generate the simulated blood glucose level readings. The simulation engine 150 receives the entered input, activity or event and the patient's parameters. The simulation engine 150 generates a simulated or estimated blood glucose level for the selected patient based on the entered input, activity, or event and the patient's parameters. Under certain operating conditions, the simulated or estimated blood glucose level is generated for a plurality of timeframes. For example, if the virtual patient software is running in the patient mode and 60 minutes has elapsed (because a user has pressed the hour advance time toolbar), the simulation engine 150 may calculate the simulated blood glucose level or simulated blood glucose level readings for the 60 minutes. For example, if the virtual patient software 105 is running in the doctor or medical professional mode, the simulation engine 150 may calculated the simulated blood glucose readings or levels for the entire simulation. In other words, the simulation engine 150 calculates a plurality of blood glucose levels for the simulation timeframe. The plurality of blood glucose levels or reading may be referred to as blood glucose data. In an embodiment of the invention, the simulation engine 150 may calculate the simulated blood glucose levels for the remaining time of the simulation. The virtual patient software 105, as described above, is able to calculate new simulated blood glucose levels based on a single input or multiple inputs and display the new simulated blood glucose levels in a variety of formats.

Under these operating conditions, the simulation engine 150 calculates a number of readings because the effect of the input, activity, or event on the patient's blood glucose level occurs over a period of time. The simulation engine 150 takes into account at least one previous reading in the calculation of the patient's simulated blood glucose levels. In an embodiment of the invention, if this is the first input, event, or activity that the virtual patient has received, the number of readings generated by the simulation engine 150 may be added to or combined with pre-existing readings or default readings for a selected timeframe. These readings may be referred to as combined blood glucose readings. If a previously generated number of blood glucose level readings have already been generated by the simulation engine 150, the previously generated number of blood glucose level readings may be added to the number of readings generated by the simulation engine 150 to create a number of combined blood glucose readings for the patient. The combined blood glucose readings may be referred to as combined blood glucose data.

The simulation engine 150 may transfer the blood glucose data to the charting and display module 110. In other words, the simulation engine 150 may transfer the plurality of combined blood glucose readings to the charting and display module 110. Illustratively, if the virtual patient software 150 is in the patient mode, the simulation engine 150 may only transfer simulated blood glucose readings for a timeframe to which the simulation has advanced. In other words, if the simulation has advanced two hours, the simulation engine may only transfer two hours of readings to the charting and display module 110. For example, if the virtual patient software is in the medical professional or doctor mode, the simulation engine 150 may transfer simulated blood glucose data for the timeframe of the entire simulation. The charting and display module 110 receives the blood glucose data (or plurality of blood glucose readings).

Depending on the mode of operation selected by the user, e.g., patient mode or doctor mode, the charting and display module 100 may display different potions or sections of the blood glucose data of the patient on a display of the computing device 100. Illustratively, if the virtual patient software 105 is in patient mode, the charting and display module 110 may only display the combined blood glucose readings for a timeframe that has been chosen by the user. For example, if an adjustment to the basal rate has been entered, in the user interface and control module 120, and transferred to the simulation engine, the simulation engine 150 generates a number of blood glucose readings based on the entered basal rate. Illustratively, if the simulation has moved sixty minutes, the simulation engine 150 generates a plurality of blood glucose readings for the sixty minutes. If the virtual patient software 105 is operating in patient mode, the charting and display module 110 may only display the combined blood glucose readings up until the period of time that the user has simulated. If the virtual patient software is in medical professional (or doctor) mode, the charting and display module 110 may display the plurality of blood glucose readings for a timeframe of the simulation. In the medical professional mode, for example, the charting and display module 110 may originally be displaying three days of blood glucose levels and this may be modified when the charting and display module 110 receives the new blood glucose data from the simulation engine.

In an embodiment of the invention, the simulation engine 150 may transfer only the number of generated blood glucose readings for the patient to the charting and display module 110 because in this embodiment of the invention, the previously generated blood glucose readings or the default/pre-existing blood glucose readings had been stored in the charting and display module 110. Thus, only the generated blood glucose data is transferred to the charting and display module. In the patient mode of the virtual patient software, the generated blood glucose data is integrated with the stored blood glucose readings in the charting and display module 110 and the charting and display module 110 displays the combined readings only up until the timeframe to which the user has advanced the simulation. In the doctor mode of the virtual patient software, the generated blood glucose data is integrated with the stored blood glucose readings and the charting and display module displays the combined blood glucose data for the timeframe of the simulation (e.g., three days). In the doctor's mode of the virtual patient software, the charting and display module 110 is displaying the effect of the input (e.g., basal rate adjustment or bolus intake) on the blood glucose level during the timeframe of the simulation.

FIG. 1(b) illustrates virtual patient software utilizing real or actual patient data according to an embodiment of the present invention. The virtual patient software 105 may include a charting and display module 110, a user interface control module 120, a storage for actual or real patient information 155, a patient parameter fit module library 160, and a simulation engine 150. In the embodiment of the invention illustrated in FIG. 1(b), the virtual patient software 105 utilizes real or actual data from the patient. The real or actual data may be received from an insulin pump, a blood glucose meter, user reported values (e.g., for meals), and/or from an insulin subcutaneous sensor. The real or actual data is input and stored in the virtual patient software 105 in an actual patient information storage 155.

In the embodiment of the invention illustrated in FIG. 1(b), the real or actual input data is transferred to patient parameter fit module 160 from the actual patient input storage 155. For example, the glucose reading from a blood glucose sensor, the insulin delivered (from a insulin pump and/or insulin shots), and/or exercise data may be sent to the patient parameter fit module 160. This means that the virtual patient software 105 adapts the parameters of the underlying metabolic model to best approximate the glucose readings of a real patient. Under these operating conditions, the patient parameter fit module 160 determines whether the patient's glucose sensor readings correspond or are similar to what the patient model expected for a patient during the measured time period. In other words, the patient parameter fit module 160 receives the actual patient data, analyzes the data, and determines what the mathematical parameters are for the patient. These determined mathematical parameters are transferred or provided to the simulation engine 150. The real or actual input data for the patient is also transferred from the actual patient data storage 155 to the user interface control module 120. If the doctor mode has been selected, the real or actual input data for the patient is transferred or sent from the user interface control module 120 to the charting and display module 110. The charting and display module 110 displays this information on, for example, graphs on a display of the computing device. Under certain operating conditions, the graphs may display blood glucose levels over time, carbohydrates consumer over time, exercise data over time, and insulin delivered to the patient over time. If the patient model has been selected, only the real or actual data up to the time the simulation has advanced to is displayed by the charting and display module 110.

A user of the virtual patient software 105 may utilize the user interface control module 110 to change or modify inputs, events, or activities. Originally, the user may have to physically input meals eaten by the patient recently. The user may review the graphs displayed by the charting and display module 110 and decide to modify one of the inputs, events, or activities, for example, carbohydrates consumed or insulin delivered to the patient. Illustratively, the user may wish to create a scenario where he or she adjusts the basal rate. After the adjustment has been made, the virtual patient software 105 simulates the patient's response in terms of blood glucose level. The adjusted input, event, or activity is transferred to the simulation engine 150 from the user interface control module 120. The simulation engine 150 receives the adjusted input, event, or activity and calculates the patient's estimated blood glucose level response to the adjusted input, event, or activity. The simulation engine 150 utilizes the parameters or constants extracted by the patient parameter fit module 160 to assist in generating the patient's estimated or simulated blood glucose level response. The blood glucose level response may be referred to as blood glucose data and may also be referred to as a number, a set, or a plurality of blood glucose readings for a period of time. The blood glucose data or the number of blood glucose readings may be transferred from the simulation engine 150 to the charting and display module 110. As discussed above, the blood glucose data may be the blood glucose data generated by the simulation engine 150. The generated blood glucose data received from the simulation engine 150 is then displayed on a display of the computing device 100 by the charting and display module 110 of the virtual patient software. In the patient mode of the virtual patient software 105, the generated blood glucose data is displayed only for the timeframe that has been entered into the virtual patient software 105. Illustratively, if the user has only run the virtual patient simulation up to 12:00 noon, only the generated blood glucose data up until 12:00 noon is displayed by the charting and display module 110. In an embodiment of the invention, the simulation engine 150 may only calculate blood glucose readings up until the 12:00 noon timeframe and this only the blood glucose readings up until the 12:00 noon timeframe may be transferred. In the medical professional or doctor mode of the virtual patient software 105, the generated blood glucose level is displayed by the charting and display module 110 for the simulation timeframe, e.g, two or three days.

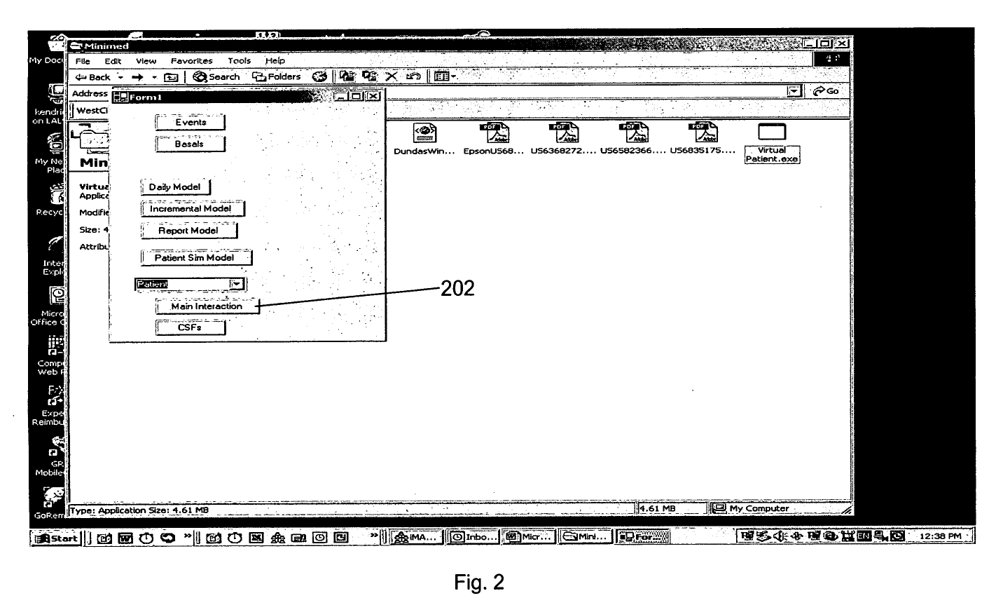

FIG. 2 illustrates an initial screen of the Virtual Patient software system according to an embodiment of the invention. The is a selection screen where a user selects to enter the Virtual Patient software and selecting a Virtual Patient software icon. In an embodiment of the system, the user may select the main interaction button 210 to enter the Virtual Patient software. The other buttons or toolbars on the initial screen may allow a system administrator to add additional features or to modify certain parameters of the Virtual Patient software. Illustratively, new events or basal options may be added to the Virtual Patient software. Additionally, different models may be added utilizing any of the four model buttons or toolbars. A user may also initiate operation of the virtual patient software by the selection of an icon on the computing device's desktop screen.

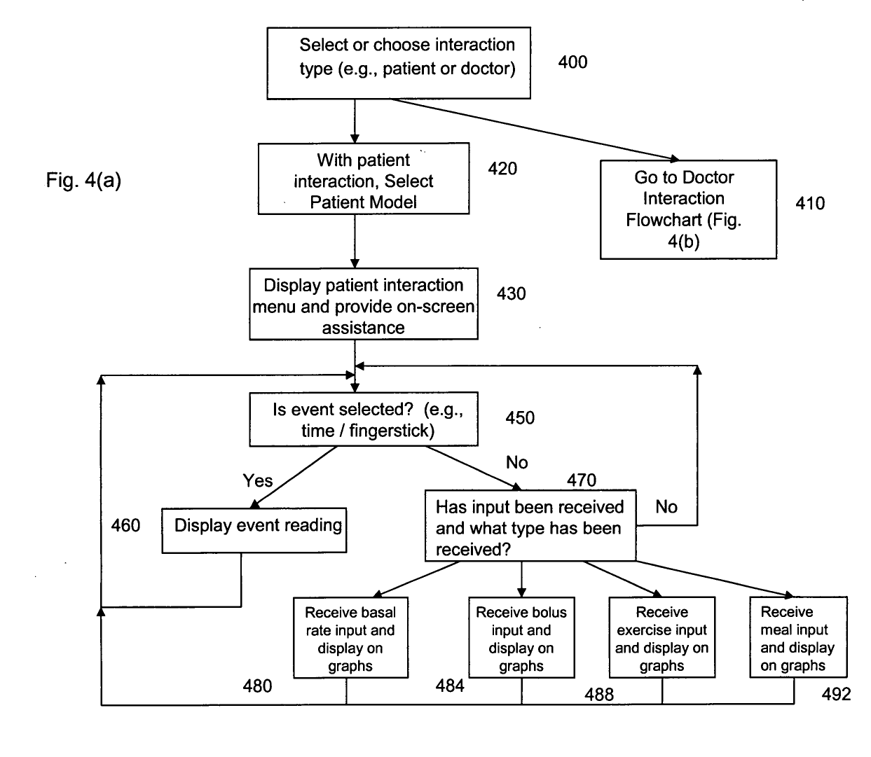

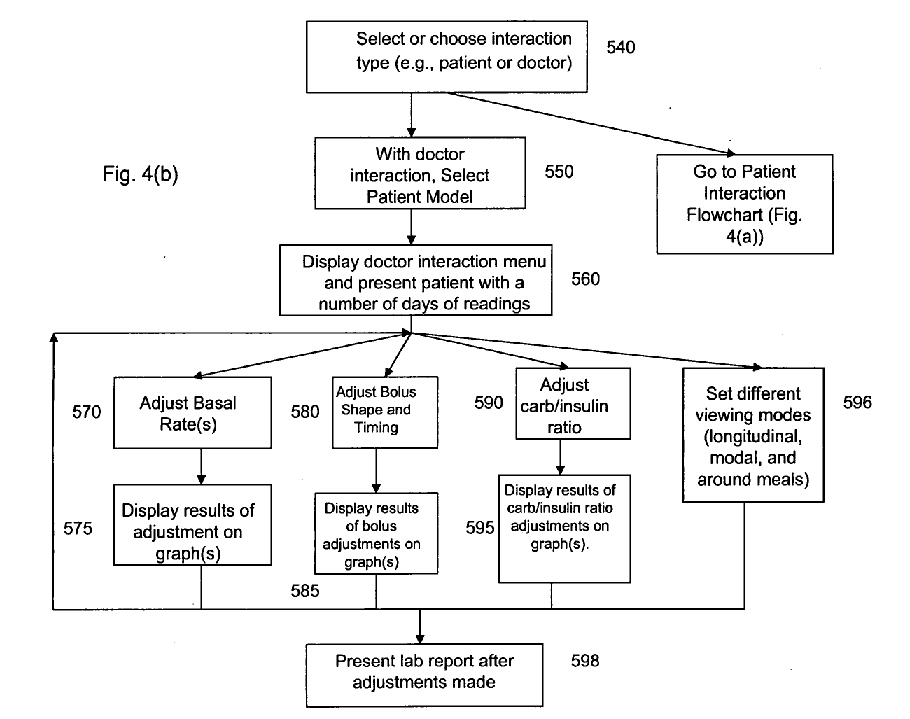

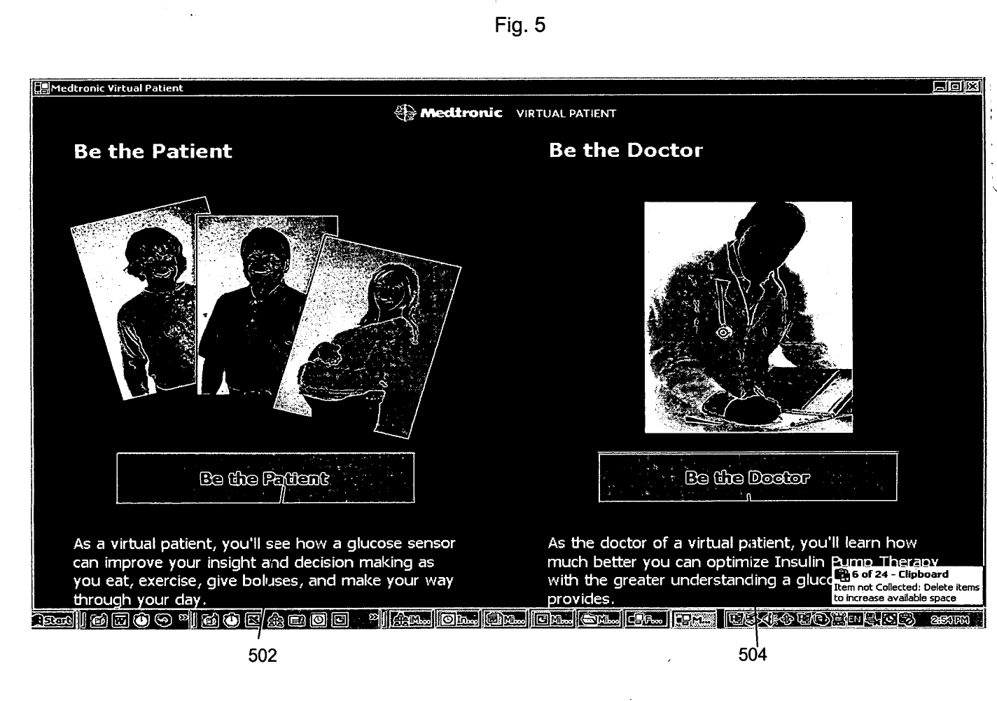

FIG. 4(a) illustrates a flowchart for a patient mode Virtual Patient software according to an embodiment of the present invention. After initialization of the software, an interaction type may be selected or chosen 400. Illustratively, the interaction type may be a doctor or patient interaction. If the doctor interaction is selected 410, a patient selection screen in the doctor interaction menu may be selected. The doctor interaction screen is illustrated in FIG. 4(b). FIG. 5 illustrates an interaction selection screen of the Virtual Patient software. In the embodiment of the invention illustrated in FIG. 5, one of the two options may be selected by the clicking of a button on the main interaction screen 500. In this embodiment, the two options presented are the “Be the Patient” option (which can be selected by clicking on the button 502) and the “Be the Doctor” option (which can be selected by clicking on the button 504). The “Be the Patient” software simulation provides a user of the Virtual Patient software 105 with the opportunity of seeing how the utilization of a glucose sensor can improve a patient's decision making by viewing the reaction of seeing the simulated patient eat, exercise, gives boluses, and progress through a sample day. The “Be the Doctor” software simulation provides a user or medical professional with an ability to learn how to optimize insulin pump therapy by seeing the results on a virtual patient of decisions that a medical professional makes in response to glucose fingerstick readings and/or glucose sensor readings. In alternative embodiments of the invention, multiple options may be presented on the menu interaction screen, e.g., (Be the Patient; Be the Medical Professional; Be the Patient utilizing the patient's own metabolic model; Be the Doctor utilizing patient's own metabolic model and actual data, etc.).

Returning to the flowchart of FIG. 4(a), if a patient interaction type is selected, a patient model type may be selected 420. Under certain operating conditions, because each individual has unique characteristics (metabolization rates, glucose creation rates, responses to exercising, etc.) a plurality of models have been created to estimate a person's reaction to certain events and medications). In an embodiment of the invention, three patient models may be available for selection. Under other operating conditions, each individual may have a patient model or model that is specifically created for the individual that estimates or predicts the individual's response to exercising, taking a bolus, eating a specific number of carbohydrates, or adjusting a basal rate of an insulin pump.

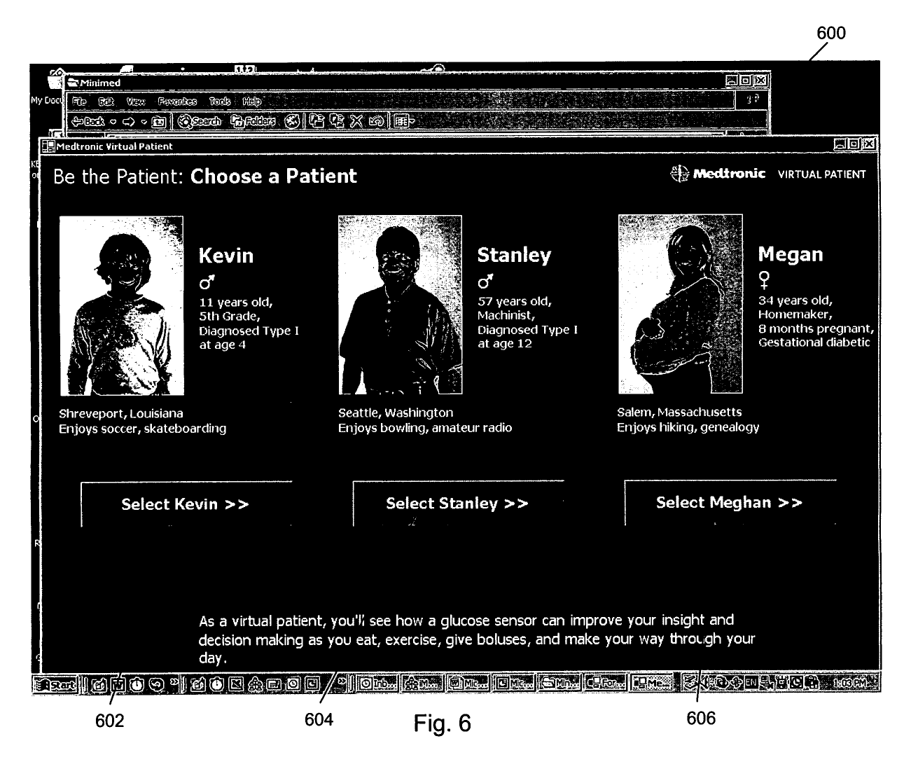

FIG. 6 illustrates a patient selection screen of the Virtual Patient software according to an embodiment of the present invention. In the embodiment of the invention illustrated in FIG. 6, one of three patient models can be selected via the patient selection screen. A user of the Virtual Patient software may select a model that is closest to the user in terms of specific characteristics, such as 1) type of diabetes; 2) age; 3) sex; and 4) other medical conditions. Each of the models or virtual patients have a different profile, history, and a response to carbohydrates (carbs) and insulin. Illustratively, the three patient models presented in FIG. 6 are Kevin, Stanley, and Megan. Kevin is a 11 year old who has Type 1 diabetes, is very athletic, and has been aware of his condition for approximately 7 years. Stanley is a 57 year old male who also has Type 1 diabetes, is physically active, and has been aware of his condition for approximately 45 years. Megan is a 34 year old female who has been diagnosed with gestational diabetes (due to her pregnancy) and is not very active at the present time even though she does enjoy walking and gardening. The patient selection menu 600 includes a menu selector or button 602 for Kevin, a menu selector or button 604 for Stanley, or a menu selector or button 606 for Megan. The patient selection menu 600 includes a start over button, icon, or selector which allows a user of the Virtual Patient software to easily return to the interaction screen to select which interaction mode they should be in (e.g., patient interaction mode or medical professional interaction mode).

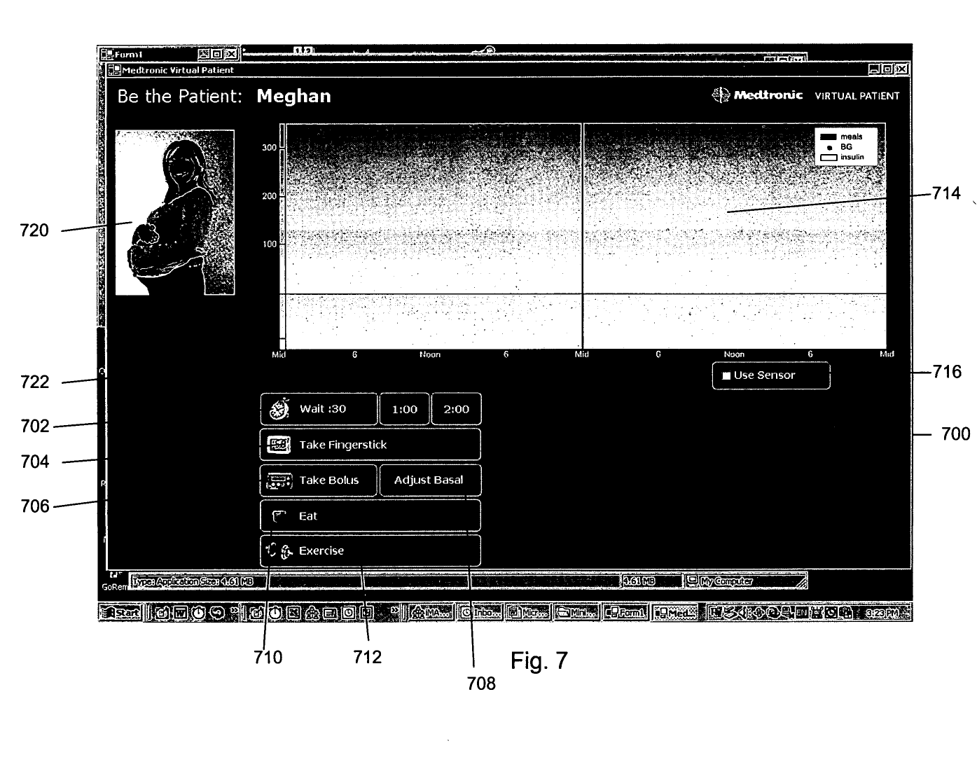

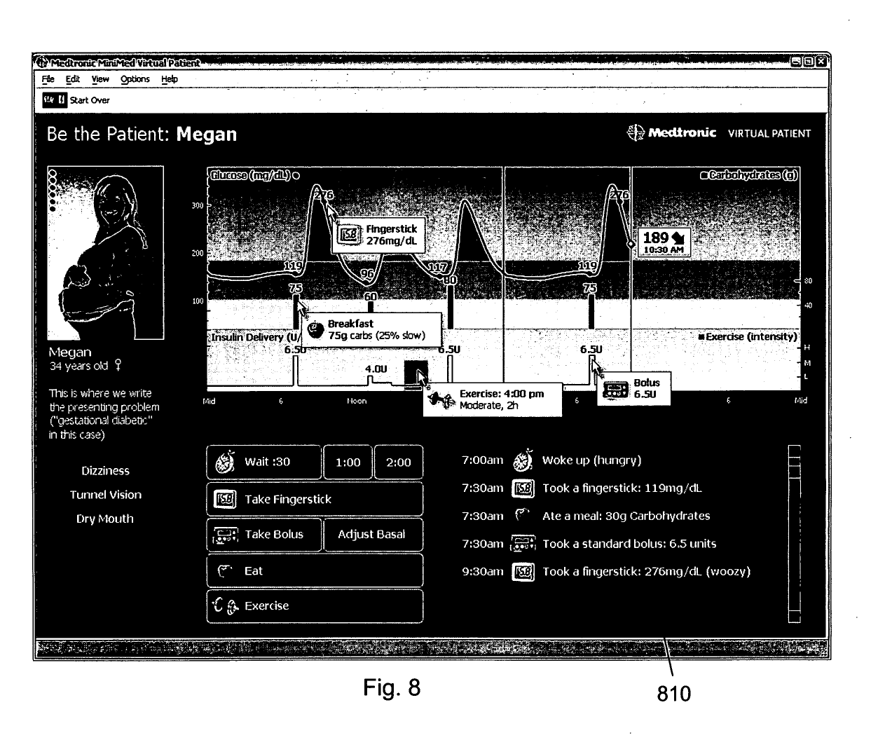

Returning to FIG. 4(a), after the patient model is selected, e.g., Megan, a patient event screen may be displayed 430. In an embodiment of the invention, a help button may be available which describes how to utilize the patient event screen. This may referred to as the introduction help screen of the Virtual patient system. FIG. 7 illustrates a patient manipulate and view screen according to an embodiment of the present invention. Accordingly to an embodiment of the invention illustrated in FIG. 7, the patient manipulate and view screen 700 may include a timing toolbar 702, a take fingerstick activation toolbar or selector button 704, a take bolus toolbar or selector button 706, an adjust basal rate toolbar or selector button 708, an eat or food intake toolbar or selector button 710, an exercise toolbar or selector button 712, and a graph display section 714. The patient manipulate and view screen 700 may also include a sensor activation toolbar or selector button 716. In an embodiment of the invention, a picture of a person representing the model may be displayed in a pane 720. Under certain operating conditions, specific symptoms of abnormal or potential problem conditions may appear below as messages in an area 722 below the pane 720 displaying an image of the person being modeled. The symptoms or potential problem conditions may be displayed because of the patient entering areas or levels on the glucose vrs. time graph that are considered potentially unsafe.

Returning to FIG. 4(a), a user of the Virtual Patient software may enter different events or enter different inputs utilizing the patient manipulate and view screen. Events may be defined as actions which cause the Virtual Patient software to display a result on the graph. For example, events may include the selection of any of the time toolbars (e.g., wait 30 minutes; wait one hour; wait two hours) or selection of the “take fingerstick” toolbar or selector button. Illustratively, inputs may be the taking bolus toolbar, the adjust basal toolbar, the eat toolbar, and the exercise toolbar. After inputs are entered, the patient model interacts with the inputs, outputs are generated, and the outputs are displayed on the graph display section 714 of the patient manipulate and view screen 700. For example, if a person exercises and selects the exercise toolbar or selector button to input the level and duration of exercise, the patient model receives the inputs and generates readings of how this affects the glucose level of the patient model over a certain time period. According to the embodiment of the invention illustrated in FIG. 7, after different events are entered, the patient model extracts an appropriate reading from the model database and presents this information on the graph display section 714 of the patient manipulate and view screen 700. For example, if the “take fingerstick” toolbar or selector button is utilized, a reading for Megan is extracted from her patient model based on the characteristics established in her model profile, and the reading is displayed on the graph display section 714.

FIG. 8 illustrates an manipulate and view screen 700 of the virtual patient software according to an embodiment of the present invention. The manipulate and view screen 700 of FIG. 8 includes an activity log 810. The activity log 810 displays all of the events and inputs that have occurred or entered for this model during a current interaction with the patient model (e.g., Megan's model). Illustratively, each time a time or time period, a fingerstick toolbar, a take bolus toolbar, or an eating toolbar is selected, the activity log 810 is updated with that information.

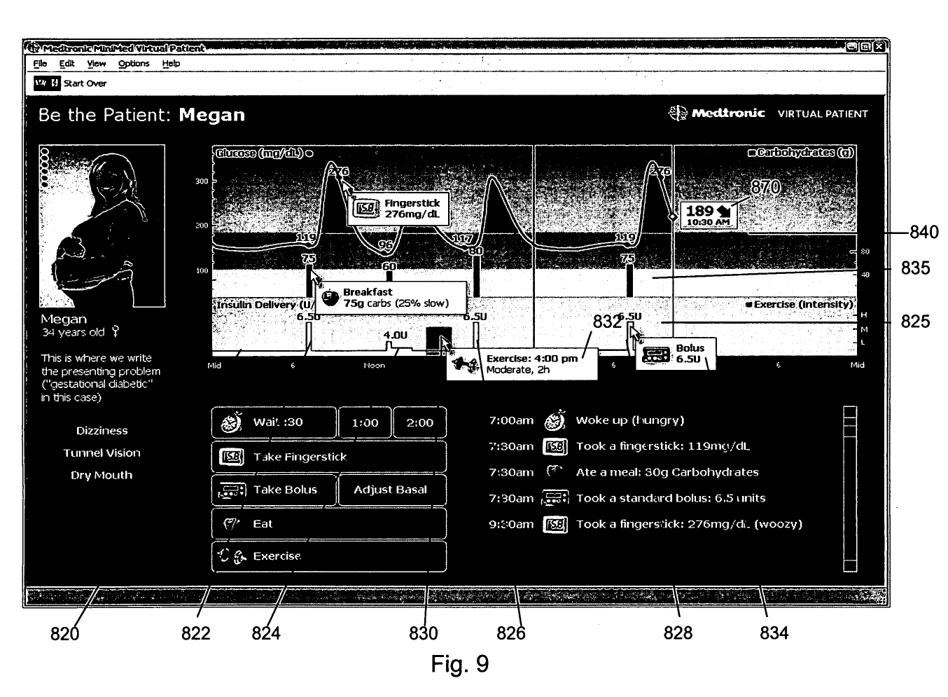

The graph display section 714 of the manipulate and view screen 700 may display a plurality of informational graphs. FIG. 9 illustrates a graph display section of a manipulate and view screen of the virtual patient software according to an embodiment of the present invention. Illustratively, the manipulate and view screen 700 of the virtual patient software includes a graph 825 illustrating insulin delivery over a two day time period. Illustratively, the graph 825 illustrates that insulin was delivered 4 times over a timeframe of a day and a half. The height of the graph represents the level of the dose of insulin input into the patient, e.g., 6.5 Units of insulin. Bolus insulin delivery is represented by reference numerals 822, 824, 826, and 828. Bolus reference numerals 822, 826, and 828 represent normal bolus intakes. Reference numeral 824 illustrates a dual wave bolus intake. Additionally, a squarewave bolus intake may be input. The graph 825 represents insulin intake from an insulin pump or a continuous insulin source by the baseline 820 of the graph 825. The graph represents exercise as a squarewave for a certain duration 830. Exercise results in the decrease of a glucose level, e.g., it absorbs or takes away the carbohydrates of a meal. The virtual patient software 105 also displays mini pop-up windows when a cursor is moved over a displayed event or input. Illustratively, in FIG. 9, when a cursor is placed over the exercise squarewave 830, a pop-up window 832 is displayed identifying that exercise occurred, when the exercise occurred, the duration of the exercise, and at what level the exercise was performed. In addition, a pop-up window 834 is displayed if a cursor is placed over one of the bolus intakes, e.g., bolus intake of 6.5 units at 7:30 am.

The graph display section 714 may provide a graph 835 that displays carbohydrates consumed by the patient at the different times of the day. For example, as illustrated in FIG. 9, 60 carbohydrates (carbs) were consumed by the patient around noon on the first day. The height of the bar represents the number of carbs consumed. In other words, the higher the bar, the higher the number of carbs. If a cursor is placed over or adjacent to one of the bars, then a mini-pop up window displays information regarding the food or meal intake by describing which meal it is, the number of grams of carbs (carbohydrates) consumed, and the absorption rate (e.g., 25% slow).

The graph display section 714 may provides a graph 840 that displays the patient's glucose level over a time period such as a two day time period. In an embodiment of the invention, the model has a pre-established timepoints in case no information is input into the patient interaction screen. For example, if Megan is chosen as the patient model and a time is selected (for example, 7:00 am), an initial reading of 119 may be read out from Megan's patient model. If the take fingerstick selector toolbar or module is selected, then a fingerstick reading appears on the graph 840. In this embodiment of the invention, a patient will not be inputting his or her own fingersticks. Instead, the patient model of the Virtual Patient software provides the fingerstick readings. The graph 840 appears as time progresses during the two day time period. In other words, for example, when a patient wakes up and sets the time to 7:00 am, the graphs 825, 835, and 840 will be drawn according to parameters supplied by the patient model. After the initial entry of time, a user may enter inputs such as eating of a meal at 7:30 am, taking a standard bolus at 7:30 am and exercising around 4:00 pm. The graph 840 is shaped by the combination of the data supplied by the patient model adjusted for the intake of insulin and carbohydrates. In other words, the inputs are entered, they are input into the patient model (simulation engine), and the patient model (simulation engine), utilizing its known characteristics and readings (patient parameters), determines a glucose reading incorporating the effect of the inputs on the glucose reading. As previously disclosed, if a cursor is placed over one of the readings (where a number is displayed), a mini pop-up window appears that displays what type of reading occurs and what the value of the reading is. Under other operating conditions, a mini pop-up window 870 appears the time of the simulation. The present time mini pop-up window 870 displays the current glucose reading, the time of the glucose reading, and an indicator of the trend of the glucose reading. For example, as illustrated in FIG. 9, the trend is that the glucose reading is trending down in a hard fashion. In an embodiment of the invention, the arrows may be either straight up or straight down. The number of arrows may correspond to a rate of glucose change, as is illustrated in the table below.

a) Explanation of Trend Arrows

| | | |
|---|---|---|
| One Arrow | ↑ Up or Down ↓ | Glucose is rising or falling at |
| | | the rate of 20-40 mg/dl over |
| | | the last 20 minutes |
| Two | ↑↑ Up or Down ↓↓ | Glucose is rising or falling at |
| Arrows | | the rate of 40 or more mg/dl |
| | | over the last 20 minutes |

In an embodiment of the invention, if the glucose sensor is activated by selecting the glucose sensor toolbar, then the patient model supplies glucose readings as if they were input from a glucose sensor. In other words, in this embodiment of the invention, a patient's actual glucose sensor is not hooked up to the Virtual Patient software and is not providing readings to the Virtual Patient software.

Returning to the flowchart of FIG. 4(a), a determination is made as to whether either an event or input has been selected in the Virtual Patient software. In FIG. 4(a), a determination is made 450 as to whether an event (such as a time input toolbar or a take fingerstick toolbar) has been selected. Note that the flowchart of FIG. 4(a) only displays one or a potential plurality of sequence flows for the software. Under other operating conditions, the virtual patient software 105 may first determine whether an input has been received. If an event has been selected, the virtual patient software displays 460 the event reading or action on a graph or multiple graphs in the graphic module of the patient interaction screen. For example, if a user has selected to wait an hour (i.e., advance an hour in time in the patient interaction), the graphs in the graph display section 714 of the patient interaction screen are updated to reflect the advancement in time of one hour. After the virtual patient software 105 displays the event reading or action on the graph, the virtual patient software 105 returns to step 450 in order to wait to determine if an additional event or input has been selected.

In no event has been selected, the virtual patient software 105 determines whether an input has been received and also what type of input has been received 470. If no input has been received, the virtual patient software returns to the input of step 450 to wait for either an input or an event. If the user has input a modification in the basal rate, the basal rate input is received, the selected patient model generates results based on the basal rate input change, and this information is displayed 480 on graph(s) in the graph display section 714. Illustratively, if a basal rate is changed, graph 825 in FIG. 8(a) is modified during the next time period by increasing or decreasing the baseline reading 820 of graph 825. Graph 840 is also adjusted according to how the modified basal rate impacts on the blood glucose level of the model patient over a period of time.

If a new bolus has been input to the virtual patient software, the new bolus is received, the patient model (simulation engine 150) receives the new bolus and generates results on blood glucose levels based on the new bolus input, and the bolus input information and the results generated by the patient model are displayed 484 on the graph display section 714 of the patient manipulate and view screen. Illustratively, if a new bolus is input to the virtual patient software, graph 825 is modified to show the time the bolus was taken and the size of the bolus. In addition, graph 840 displaying the blood glucose level of the selected patient is modified to show the effects of the taking of the bolus over a period of time.

If an exercise input has been input to the virtual patient software, the new exercise input has been received, the patient model (simulation engine 150) receives the new exercise input and generates results on blood glucose levels based on the new exercise input, and the exercise input information and the results generated by the patient model are displayed 488 on the graph display section of the patient manipulate and view screen. Illustratively, if an exercise input is received by the virtual patient software, graph 825 is modified to show the time, the duration, and the level of the patient's exercise. In addition, graph 840 is modified to show the effects of the patient exercising over a period of time.

If a meal input has been input to the virtual patient software, the meal input is received, the patient model receives the new meal input and generates results on blood glucose levels based on the meal input, and the exercise input information and the results generated by the patient model are displayed 492 on the graph display section of the patient manipulate and view screen. In addition, the virtual patient software 105 provides for the generation of the appropriate bolus that a patient needs to take in order to counteract the effects of the number of carbohydrates eaten during a meal. Illustratively, if an exercise input is received by the virtual patient software, graph 835 is modified to show the meal, how many carbohydrates were estimated to be present in the meal, and how easily the carbohydrates are digested. In addition, graph 840 is modified to show the effects on the blood glucose level of the patient over a period of time, after the patient has ingested the entered meal.

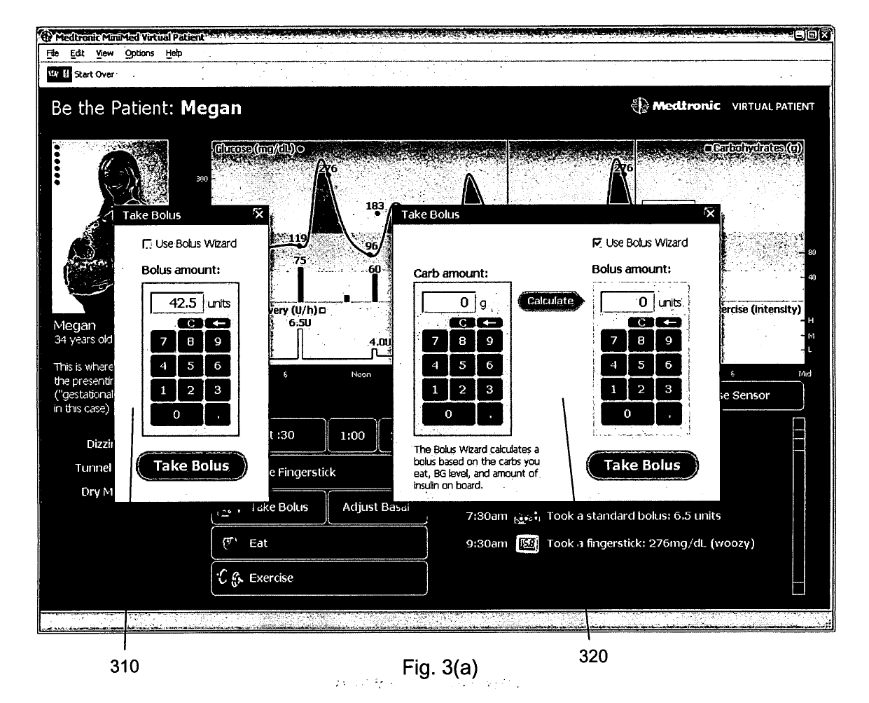

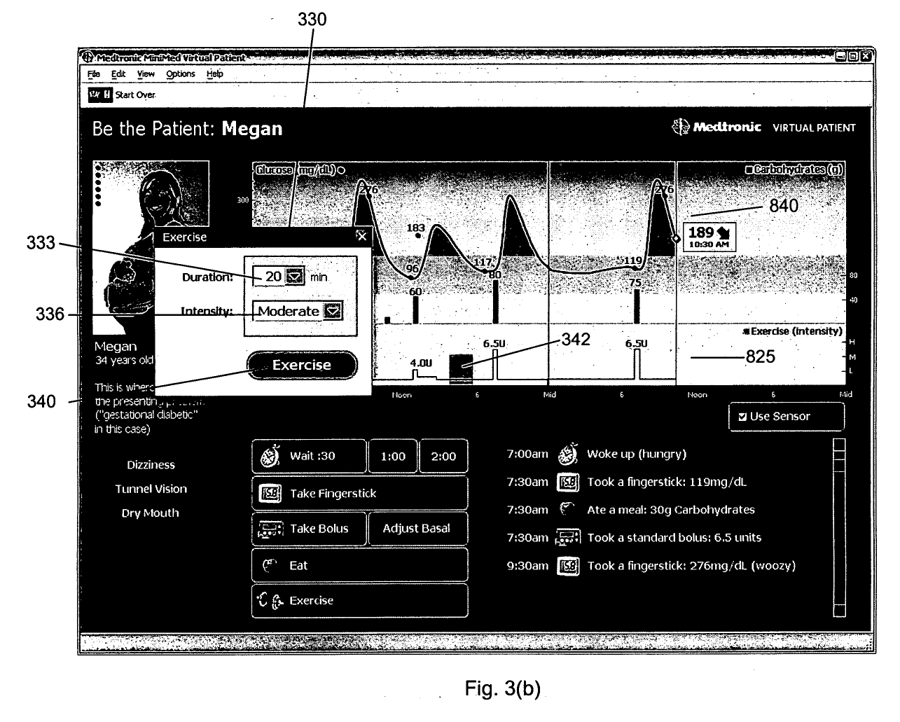

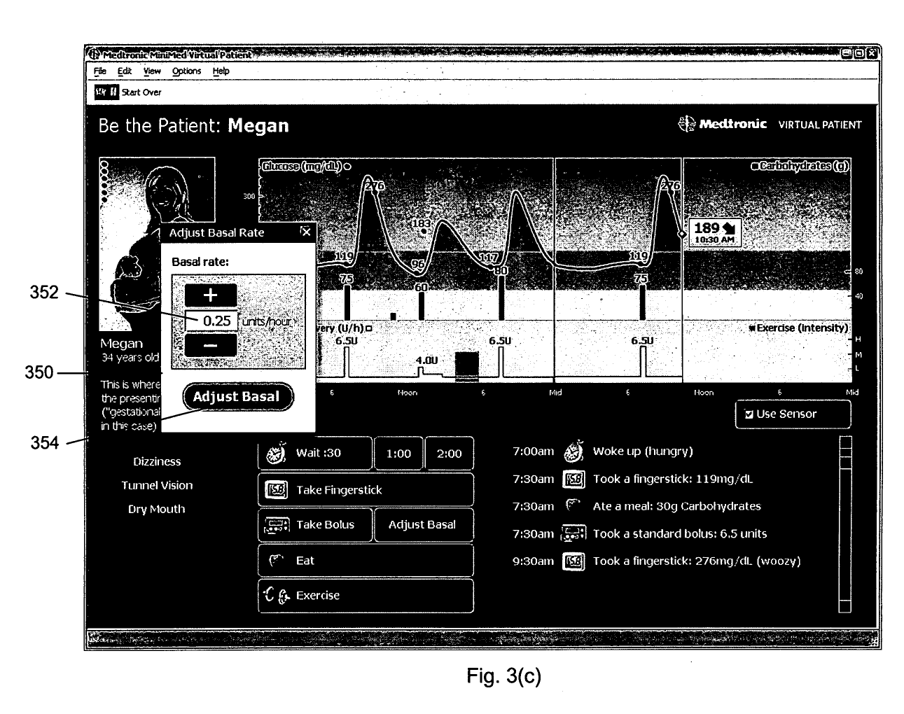

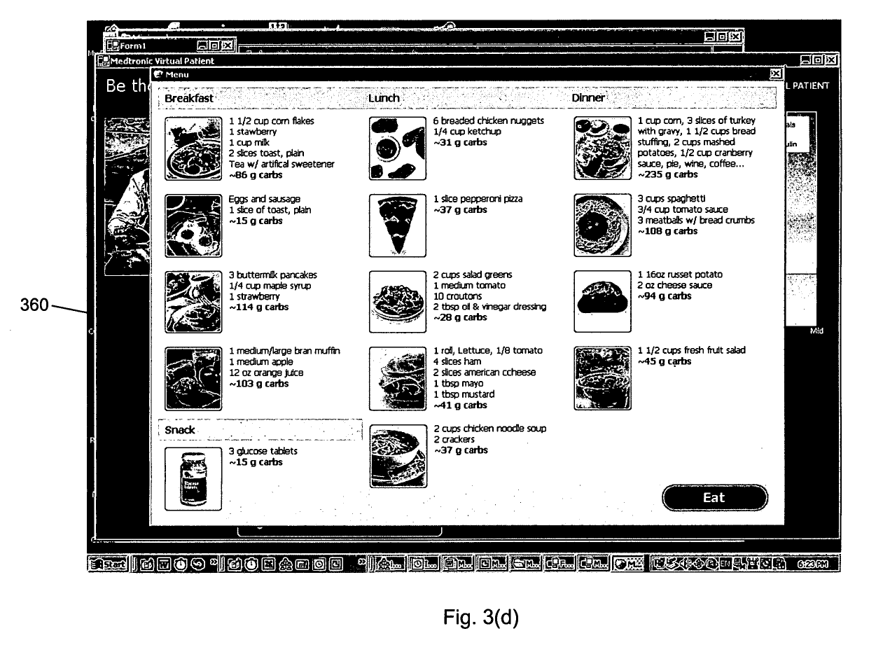

FIG. 3(a) illustrates a bolus input window and a bolus wizard window according to an embodiment of the present invention. In an embodiment of the invention, the bolus input window 310 allows a user of the virtual patient software 105 to input a bolus amount (in terms of insulin units). The virtual patient software 105 enters this information into the selected patient model (or simulation engine 150). The results of the entered bolus on the patient model's blood glucose level are calculated and displayed on graph 840. In addition, the insulin delivery graph 825 is updated to included the newly added bolus. Under certain operating conditions, the graph 825 and the graph 840 are updated after the user has selected a new timeframe. Specifically, under these operating conditions, if the bolus is taken at 11:30 am and the graph is only displaying the graphs up to the 11:30 am timeframe, then graphs 825 (blood glucose level) and 840 (insulin delivery graph) are not updated until a new timeframe is selected.

The bolus wizard window 320 allows a user of the virtual patient software to determine the bolus units to be input or paired based on the carbohydrates a patient has consumed during a meal timeframe. A user of the virtual patient software may enter the number of carbohydrates into the bolus wizard window 320 (i.e., the number of grams), press the calculate selector button or calculate toolbar, and the bolus amount that the virtual patient software determines counteracts the carbohydrates consumed is presented. Under certain operating conditions, the selected patient model takes into consideration that a user often underestimates and overestimates the number of grams of carbohydrates that a patient consumes.

FIG. 3(b) illustrates an exercise input screen in a patient manipulate and view menu according to an embodiment of the present invention. In an embodiment of the invention, the exercise input screen 330 is a mini pop-up window. The exercise input screen 330 includes a entry window 333 where a duration of an exercise can be input along with a second entry window 336 where a level of intensity for the exercise (e.g., low, medium, or high) is input. Under certain operating conditions, the entry window 333 and the second entry window 336 may be implemented as drop-down menus. The longer the amount of exercise or the higher the intensity of the workout generally means that a patient's blood glucose level rises as the exercise is being completed or after the exercise has been completed. The exercise input screen 330 also includes an exercise input button 340. The exercise input button 340, when clicked or selected, results in the inputs entered in the entry window 333 and the second entry window 336 being entered into the patient model (or simulation engine 150). The patient model or simulation engine 150 calculates the effects of the entered exercise duration and intensity on the patient's blood glucose level. The adjustment to the blood glucose level caused by the entered exercise input is displayed on graph 840 in the graph display section of the patient manipulate and view screen. In addition, the input exercise is presented or displayed on the insulin delivery graph 825, as illustrated in FIG. 3(b) by the green rectangle shape 342.

FIG. 3(c) illustrates a basal adjustment rate menu in a patient interactive manipulate and view screen according to an embodiment of the invention. In an embodiment of the invention, the basal rate adjustment menu 350 may be a pop-up menu or a menu superimposed on the patient manipulate and view screen. The basal rate adjustment allows an adjustment of the basal rate in an input window 352 by either entering a value or by pressing a “+” or a “−” button. After the desired basal rate has been selected, an adjust basal rate button 354 is selected or chosen and the basal rate is entered into the selected patient model. The selected patient model receives the adjusted basal rate and calculates the effect of the adjusted basal rate on the patient's blood glucose level. The virtual patient software 105 displays the results on the patient's blood glucose level over a time period in the blood glucose level graph 840. The virtual patient software also may display the adjusted basal rate on the insulin delivery graph 825. Under certain operating conditions, the adjusted basal rate is only displayed on the insulin delivery graph 825 at a time after the basal rate has been adjusted.

FIG. 3(d) illustrates a carbohydrate determination menu according to an embodiment of the present invention. In an embodiment of the invention, a user may just enter the grams of carbohydrates, if this information has been provided, for example, if a pre-packaged meal is or has been consumed. In the embodiment of the invention illustrated in FIG. 3(d), the carbohydrate determination menu 360 is displayed when a user selects the eat toolbar on the patient manipulate and view menu. A plurality of different meal and/or snack combinations are presented in the carbohydrate determination menu 360. The meal and/or snack combinations may represent a large number of potential standard patient meals to enable the patient or user to determine a carbohydrate value of a recently eaten meal (or a meal that will be eaten). Under certain operating conditions, a carbohydrate count for two meal selections may be the same or very similar. However, some of the carbohydrates may be slower to digest and may have a slower or faster effect on the patient's blood glucose level. After a meal and/or snack combination is selected, the selected patient model of the virtual patient software takes into consideration not only the number of grams of carbohydrates, but also whether or not the carbohydrates are slow-acting or fast-acting. After either the number of carbohydrates has been entered or the meal has been selected, the virtual patient software determines the effect of the ingested carbohydrates on the selected patient's blood glucose level and displays the resulting blood glucose level on graph 840. In addition, the number of grams of carbohydrates is displayed on graph 835 of the graph display section.

FIG. 4(b) illustrates a flowchart of a doctor interaction model of the virtual patient software according to an embodiment of the present invention. After the doctor or medical professional interaction type is selected 540, a patient model may be selected 550. In the embodiment of the invention of FIG. 4(b), a doctor interaction menu is displayed and a patient corresponding to the selected patient model is presented 560. Presenting of the patient is the displaying of an initial overview of the condition of the patient of the patient model and also displaying a number of days of data for the patient. Under certain operating conditions, the displaying of the initial overview is in the foreground and the displaying of the number of days of data for the patient is in the background.

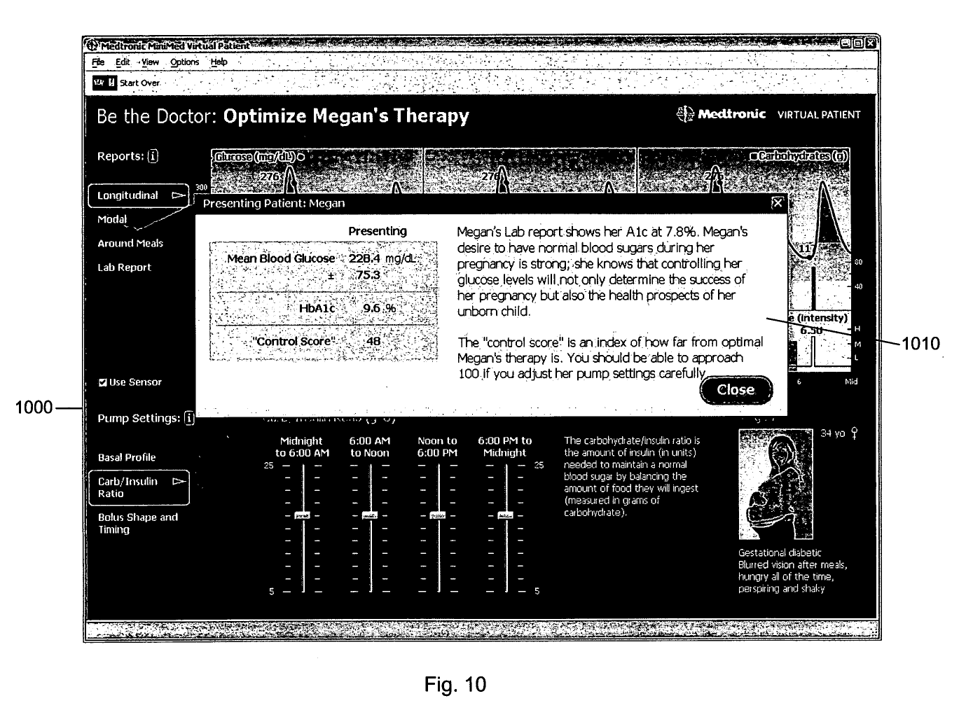

FIG. 10 illustrates a presenting screen 1000 for a patient, e.g., Megan, in the doctor interaction mode of the virtual patient software according to an embodiment of the present invention. In the presentation pop-up menu 1010, a patient's statistics may be displayed. These statistics are based on a number of days of data, e.g., three days of data. In an embodiment of the invention, the statistics have been pre-stored in the patient model. In an alternative embodiment of the invention, the statistics may have been input from an outside source. If a patient is wearing a glucose sensor which has storage capabilities, such as the Medtronic glucose sensor, a number of days of readings from the sensor may be loaded into the patient readings database so that a patient's actual data is utilized by the medical professional in the medical interaction mode of the virtual patient software 105. Statistics may, but are not limited to, a mean blood glucose reading over the presenting time frame, a HbAlc percentage, a deviation from the mean blood glucose reading, and a control score, which is an index of how far from an optimal therapy Megan's therapy is. Under certain operating conditions, the optimal control score is 100 if all the settings are adjusted in the most efficient matter. The presenting patient menu may also provide a brief interpretation of the patient's condition.

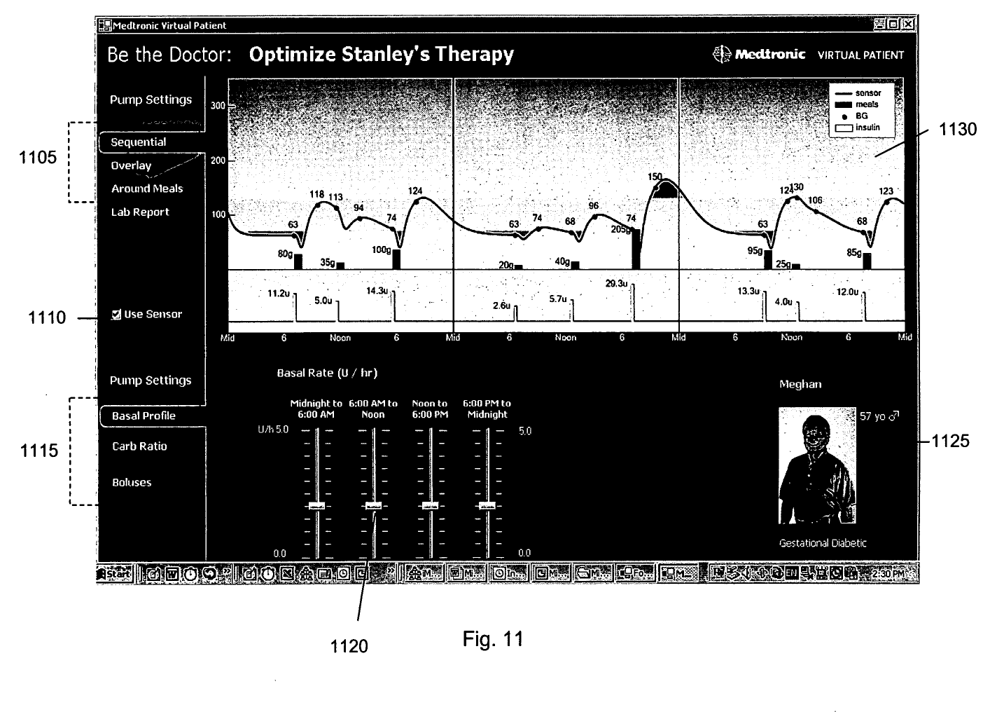

FIG. 11 illustrates a doctor interaction manipulate and view screen according to an embodiment of the present invention. The doctor interaction manipulate and view menu includes a display orientation menu 1105, a display sensor checkbox 1110, a modify input or pump settings menu 1115, an actual adjustment toolbar 1120, an image profile 1125, and a graph display section 1130. In the embodiment of the invention illustrated in FIG. 11, the display orientation menu 1105 may allow selection of a sequential display of information (e.g., three days sequentially), an overlay or modal display of information (e.g., the graph covers a one day timeframe and all three days readings are superimposed on the one day timeframe), and an around means display of information, e.g., (where each meal is displayed in a separate mini menu and three days of data around each of the meals is displayed in the separate mini-menu).

The display sensor checkbox 1110 allows for a user of the Virtual Patient system to display continuous or periodic sensor reading on the graph display section 1130 of the doctor manipulate and view screen. If the display sensor checkbox 1110 is not selected, then only datapoints corresponding to the fingerstick readings are displayed in the graph display section 1130. The image profile 1125 presents a picture of the patient corresponding to the selected patient model. A modify input or pump settings sub-menu 1115 allows the selection of different basal rates, the setting of a different carb/insulin ratio, and the inputting of specific boluses and the bolus timing. The actual adjustment toolbar 1120 allows for the setting of the different basal rates during different time ranges of the days, the setting of a different carb/insulin ratio during different time ranges of days, and the inputting of different bolus and when the boluses are going to be taken by the patient. The graph display section 1130 displays a plurality of graphs depending on the display orientation menu selection of the doctor manipulate and view menu 1130.

Returning to FIG. 4(b), from the doctor interaction menu, a user can select the basal profile option and adjust 570 the basal rates for different time periods. After the basal rates have been adjusted, the virtual patient software 105 calculates the effects of the adjusted basal rate(s) on the glucose level and displays 575 the results of the adjusted basal rates on the graph display section 1130 along with the increased or decreased basal rate. From the doctor interaction manipulate and view screen, a boluses option may be selected and a bolus input screen may be displayed.

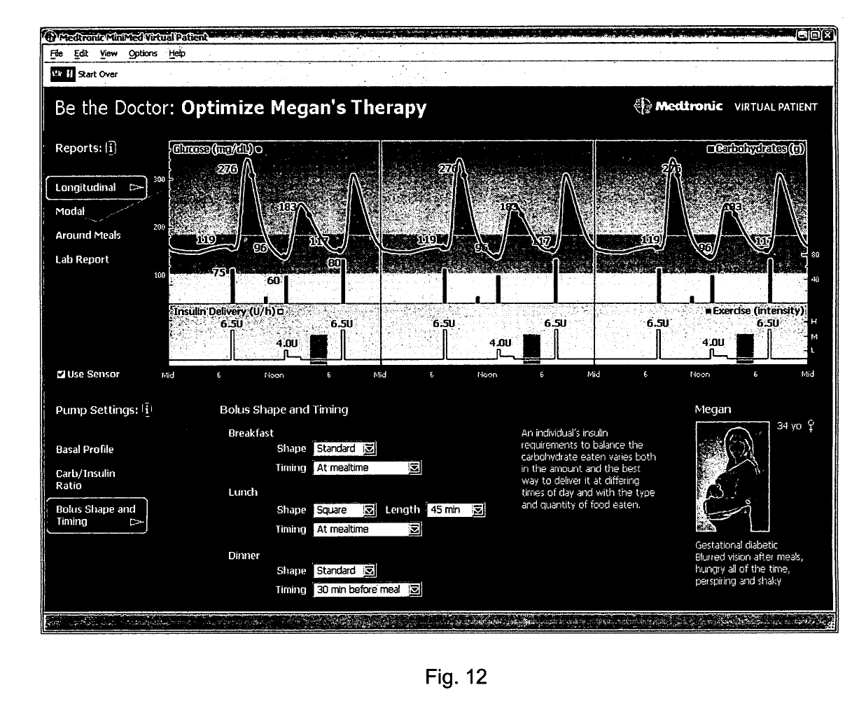

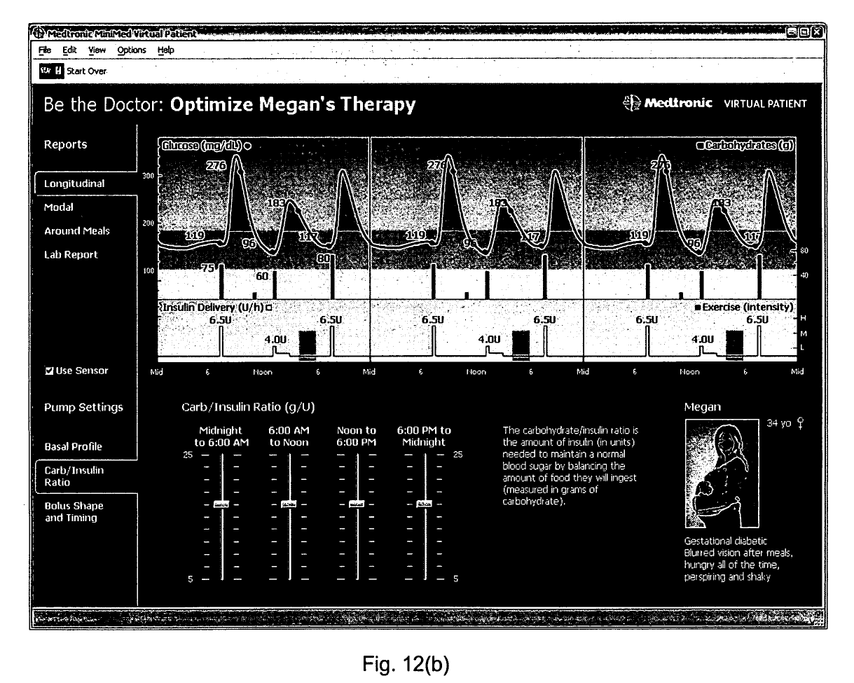

FIG. 12 displays a doctor interaction menu including a bolus input screen according to an embodiment of the present invention. In an embodiment of the invention, three boluses may be entered, with each bolus corresponding to a meal time. The shape of the bolus may be entered along with the timing of when the bolus was taken. If the bolus is of a specific type, a user may also input the length of time for how long the bolus takes to enter the patient's system.

Returning to the flowchart of FIG. 4(b), the virtual patient software may already have standard or pre-existing bolus inputs, but the virtual patient software allows for the selection 580 of the bolus shape and the timing (e.g., around mealtime, 30 minutes after a meal, etc.). The virtual patient software takes the input bolus information and applies this information to the selected patient model and generates a resulting graph (e.g., resulting datapoints) of how the blood glucose level selected patient model would respond to the adjusted input bolus information. The resulting graph is displayed 585 in the graph display section 1130 of the doctor manipulate and view screen. In addition, the adjusted bolus information is also displayed on an insulin delivery graph in the graph display section 1130.

From the doctor interaction manipulate and view screen, a insulin/carbohydrate ratio may also be adjusted. The carbohydrate/insulin ratio may be adjusted for different time periods. Because the insulin/carbohydrate ratio in real patients can differ throughout the day, this function allows pump users to account for this as they program their insulin pump. FIG. 12(b) illustrates a doctor manipulate and view screen where the adjust carb/insulin ration has been selected. In an embodiment of the invention, a 24 hour day may be broken up in to four six hour periods that each can have a different carbohydrate/insulin ratio. After the user adjusts the carbohydrate/insulin ratio for the selected time periods, the results of the adjusted carb/insulin ratio on the blood glucose level are displayed 595 in the graph display section 1130 of the doctor manipulate and view screen. In addition, on a insulin delivery graph in the graph display section, the carbohydrates and insulin are displayed in relation to each other. For example, if the carb/insulin ratio is 6 to 1, then 72 grams of carbohydrates has the same height as 12 units (Us) of insulin on the insulin delivery graph.

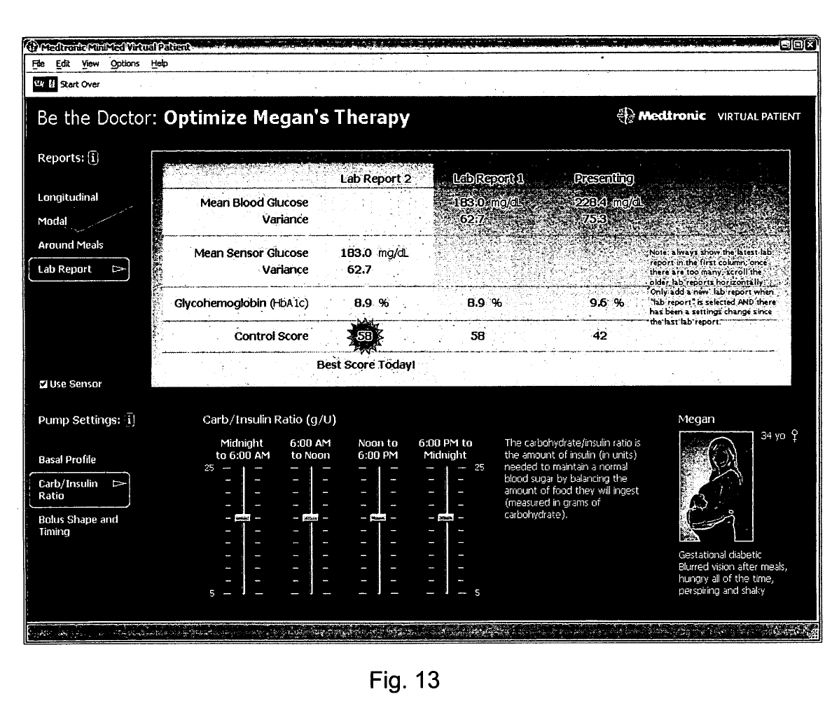

The virtual patient software 105 also includes different viewing modes (longitudinal, modal and/or around meal display modes). FIGS. 13(a), 13(b), and 13(c) display longitudinal, modal, and around meal display modes selectively. A user may select the display mode by pressing one of the selection or options in the display orientation menu 1105.

After any of the inputs have been adjusted (basal rate, bolus shape and timing, and carb/insulin ratio), or after the display orientation has been selected or adjusted, the process or the virtual patient software returns to the output of step 560. In other words, after the viewing mode is changed, for example, a user of the virtual patient software can return to adjust the basal rate. Similarly, the user can adjust the bolus shape and timing. This is illustrated in FIG. 4(b) by the return links to the output of box 560.

After the medical professional has made all the necessary adjustments that are desired to be made to the selected patient model, the medical professional may desire to run 598 a lab report for the selected patient model. In other words, the medical professional would like to see a report that details how the modifications that were made to the patient's therapy performed in terms of overall statistics. FIG. 13 illustrates a lab report displayed in a patient interaction screen according to an embodiment of the present invention. In an embodiment of the invention, the lab report may be displayed as a pop-up window and take the place of the graph in the graph display section of the doctor interaction screen. The lab report shows the current performance of the patient in regard to the selected patient model with the optimization (or adjustments) that the user, i.e., the medical professional, has made. Under certain operating conditions, multiple lab reports can be run on a patient during operation of the virtual patient software. Lab reports may be run when the lab report selection button is selected and there has been a settings adjustment or change since the running of the previous lab report. The lab report provides a control score to guide the medical professional in determining the best treatment for the patient model.

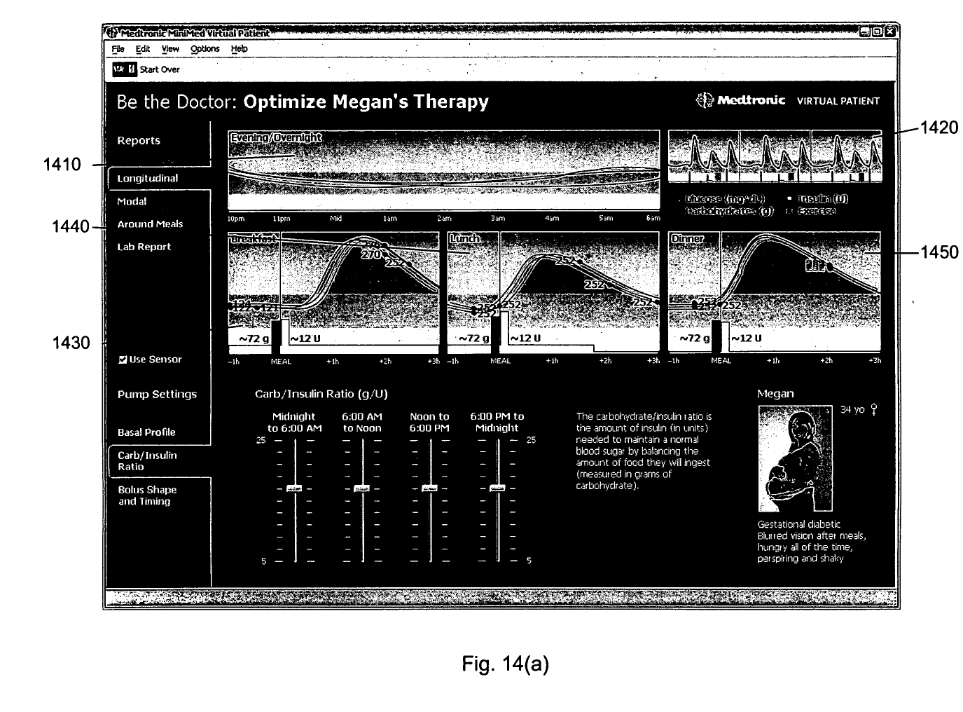

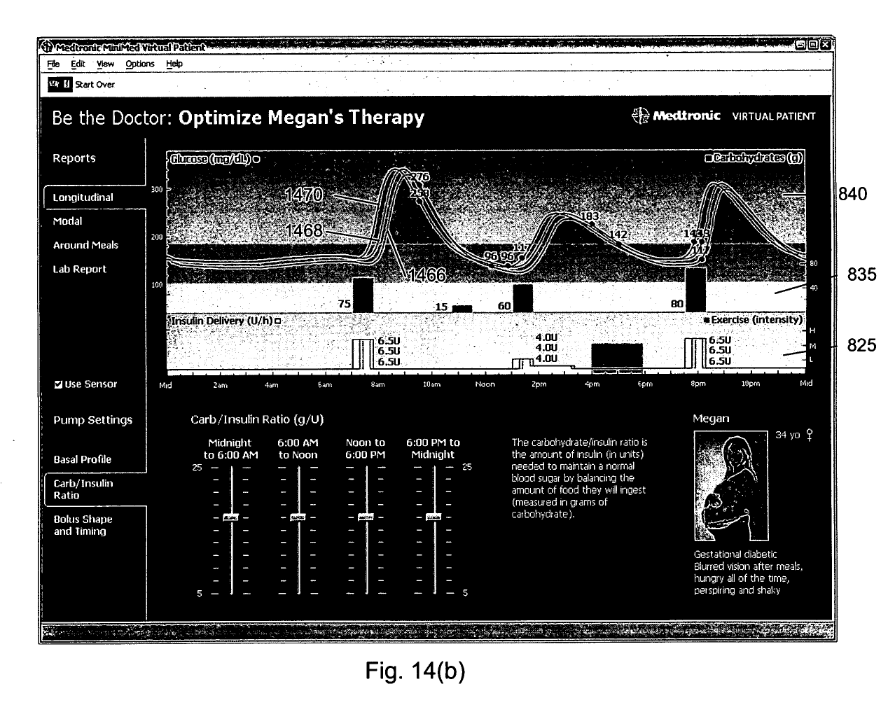

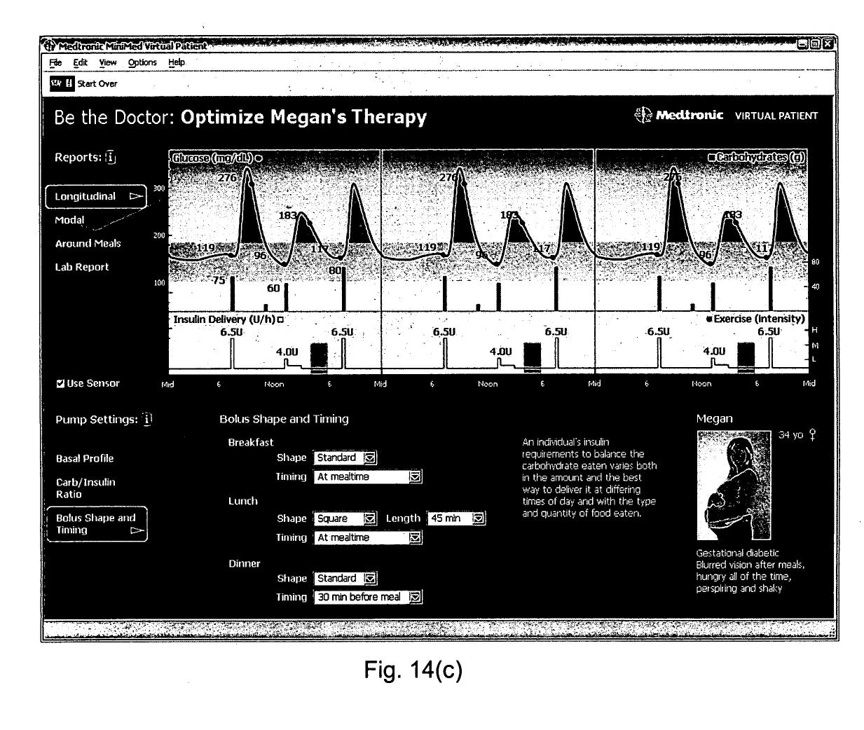

FIG. 14(a) displays a doctor interaction manipulate and view screen being displayed in an around meals view according to an embodiment of the invention. In the embodiment of the invention illustrated in FIG. 14(a), five different graphs are displayed in the around meals view on the doctor manipulate and view menu. The doctor manipulate and view menu in the around meals mode includes an evening/overnight submenu 1410, a blood glucose multiple day submenu 1420, a breakfast submenu 1430, a lunch submenu 1440, and a dinner submenu 1450. The evening/overnight submenu 1410 displays the blood glucose level of the selected patient during the evening hours, e.g., 10 pm to 6 am. The blood glucose multiple day submenu 1420 displays readings for multiple days (e.g., 3 days). The readings include the selected patient's glucose level, the insulin delivered to the selected patient, the carbohydrates ingested by the selected patient, and the exercise input for the selected patient. The multiple day submenu 1420 is similar to the longitudinal view of the doctor manipulate and view menu of the virtual patient software. The breakfast submenu 1430 displays blood glucose levels for a timeframe around breakfast over a number of days. The breakfast submenu 1430 also displays the carbohydrates consumed and the insulin delivered to the selected patient at meal time and for the timeframe around breakfast. In an embodiment of the invention, the carbohydrates total is shown in scale to the corresponding bolus that is taken to combat the effect of the patient eating the meal or carbs.

The lunch submenu 1440 and the dinner submenu 1450 include similar displays to the breakfast submenu 1430 except these menus display blood glucose levels, carbohydrates consumed, and bolus input units around the lunch timeframe and the dinner timeframe, respectively.

FIG. 14(b) displays a doctor manipulate and view screen being displayed in a modal view according to an embodiment of the present invention. The doctor manipulate and view screen includes a insulin delivery graph 825, a carbohydrate ingested graph 835, and a blood glucose level graph 840. The timeframe graphed in the doctor manipulate and view screen being displayed in a modal mode is one day. Each of the days having readings displayed in the doctor manipulate and view screen are displayed in a different color or with a different width/typeface. Illustratively, line 1466 represents Monday, line 1468 represents Tuesday, and line 1470 represents Wednesday. This view allows a doctor utilizing the virtual patient software to see multiple days of readings for a specific patient and to determine if a timeframe specific problem is occurring.

FIG. 14(c) illustrates a longitudinal view of the doctor view and manipulate menu according to an embodiment of the invention.

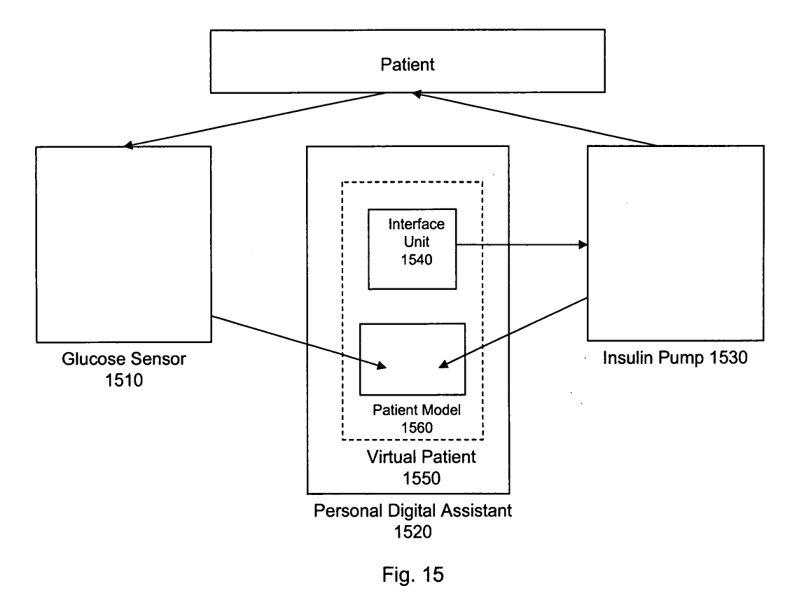

FIG. 15 illustrates a closed-loop system including a glucose sensor, a computing device having virtual patient software, and an insulin pump according to an embodiment of the present invention. Although FIG. 15 illustrates that the glucose sensor 1510, the virtual patient software computing device (e.g., a personal digital assistant 1520), and the insulin pump 1530 are separate devices, in alternative embodiments of the invention, the three devices may be combined into one device (i.e., a combination glucose sensor/insulin pump having a memory that can store and execute the virtual patient software 1550 along with a display that can present a user of the system with a graph). In an alternative embodiment of the invention, the glucose sensor 1510 and the insulin pump 1530 may be combined in a single device and the virtual patient software 1550 is installed on a portable computing device, e.g., a portable digital assistant 1520, a portable telephone, a blackberry, or a laptop. The three devices may be physically attached via communication cables that communicate utilizing parallel, serial, or Ethernet communication protocols. In alternative embodiments of the invention, the devices may communicate with each other via wireless communications utilizing wireless communication protocols such as Bluetooth or any of the IEEE 802.11 wireless communication protocols. For example, the glucose sensor 1510 may transmit glucose readings for a patient by Bluetooth to the virtual patient software 1550 on a personal digital assistant 1520. The virtual patient software 1550 may include a patient model 1560 and a user interface unit 1540.

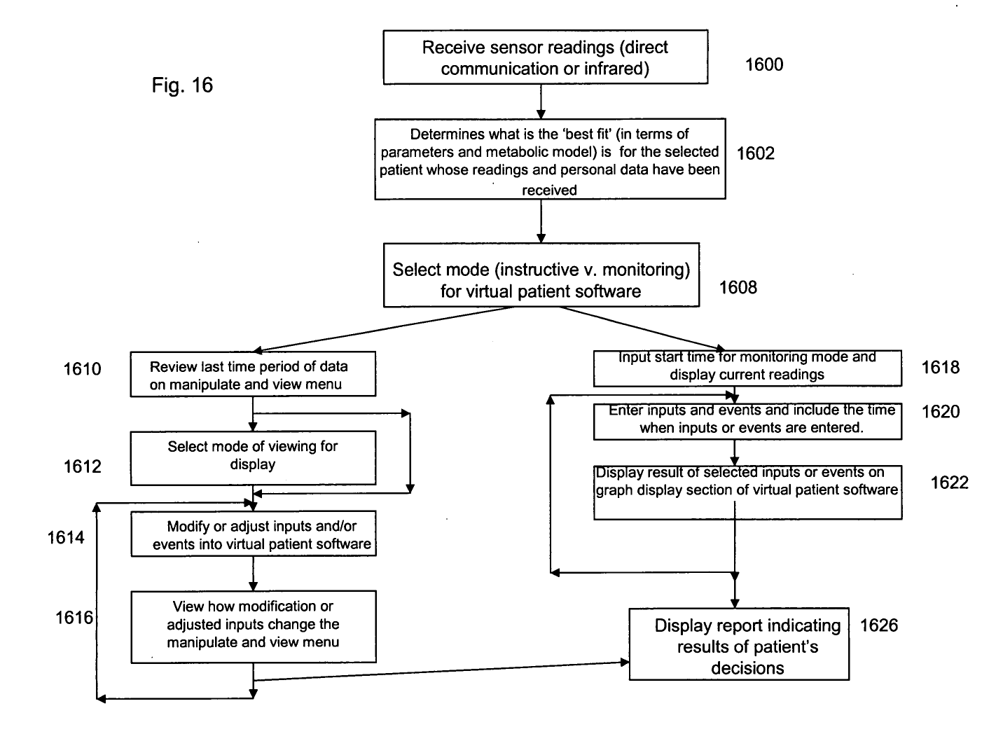

FIG. 16 illustrates a flowchart of operation for customized virtual patient software according to an embodiment of the present invention. The virtual patient software 105 may receive 1600 sensor readings for a selected patient from a glucose sensor. The virtual patient software may receive glucose sensor readings on a continuous basis, a periodic basis, e.g., every few hours, or on a batch basis, e.g., loading a number of days of readings into the virtual patient software. The glucose sensor readings may be transmitted over a communication cable, wirelessly, or via a portable memory device (such as a memory card, a memory stick, a portable hard drive, a floppy disk, a CD, or a DVD). The software will require additional personal information, (such as weight, how long patient has had diabetes, patient's lifestyle, patient's dietary habits, etc.) in order to create the virtual patient model.

The virtual patient software determines 1602 what the ‘best fit’ is for the selected patient whose readings and personal data have been received. This means that the software adapts the parameters of the underlying metabolic model to best approximate the glucose readings of a real patient. Under these operating conditions, the patient model (patient parameter fit module 160) may be determining whether the patient's glucose sensor readings correspond or are similar to what the patient model (patient parameter fit module 160) expected for the patient during the measured time period.

If no metabolic model can ‘fit’ the patients glucose data, the virtual patient software 105 is not able to simulate the glucose metabolism of this particular patient. In an alternative embodiment of the present invention, a patient model may receive the glucose sensor readings directly, outside of the virtual patient software 105, and after the patient model is created external to the virtual patient software 105, the newly created patient model may be imported into the virtual patient software with patient parameters being placed in the patient parameter fit module and any mathematical algorithms or logic being provided to the simulation engine 150.

The mode of utilization for the virtual patient software may be selected 1608. In an embodiment of the invention, the modes may be an instructive mode (educational mode) versus a monitoring mode. Generally speaking, in the instructive mode, a patient or medical professional may view results of selected therapies on the selected patient's model. In this mode, the results are not actually what would appear on the patient's glucose sensor. Instead, it is an estimation or prediction of what may occur if a specific therapy or event occurs (e.g., changing a basal rate or eating a meal).

If the instructive mode has been selected and the virtual patient software has received the sensor readings from a blood glucose sensor, the virtual patient software may display 1610 a pre-determined period of time and the corresponding sensor readings. In an embodiment of the invention, the virtual patient software 105 may display the last three days of sensor readings along with any other events or inputs that the patient may have entered (i.e., meals ingested, boluses took, insulin shots took). Under certain operating conditions, the virtual patient software 105 may also be receiving information from the insulin pump, (e.g., regarding information such as a basal rate). In some embodiments of the invention, because blood glucose sensor readings from the blood glucose sensor may be the only information supplied to the virtual patient software, the graph display section 714 of the manipulate and view menu may only display a blood glucose level graph (e.g., graph 840) and not the other two graphs (because no data has been into the patient model regarding either the carbohydrates consumed or the insulin delivered to the patient).

Under certain operating conditions, a user may decide to change or modify 1612 the viewing orientation or viewing mode for the virtual patient software. Illustratively, a longitudinal mode may be selected by default, but the user may change this viewing mode to an around meal mode or a modal mode (where one 24 hour timeframe is graphed or displayed and each of the selected days are displayed one on top of the other).

Events may be entered into the virtual patient software or inputs such as basal rates, meals, or bolus units taken may be entered 1614 into the virtual patient software 105. If events are input, the virtual patient software 105 responds to these events. If the event is the entering of a time, such as utilizing the time input toolbar on the manipulate and view screen, the virtual patient software 105 may modify the information shown on the graph display section 714 to only incorporate a newly selected timeframe data from the last selected timeframe data. For example, if a new time is selected (e.g., 3:00 pm) and the previously selected time was 12:00 noon, the virtual patient software may only display information for the 3 hours between 12:00 noon and 3:00 pm. Illustratively, this is utilized in the patient mode.

Under certain operating conditions, because this is instructive mode, the time toolbar may be disabled because the instructive mode is established as a teaching tool, and may not be utilized for real-time monitoring.

In an embodiment of the invention, the event to be input may be a “take fingerstick” event where the user/patient is actually taking a fingerstick reading. Because a blood glucose sensor is also being utilized to measure the blood glucose level in a patient, the fingerstick event may be utilized to calibrate the blood glucose sensor. In other words, the virtual patient software 105 may ask a user for a fingerstick reading, receive the fingerstick reading, and compare the fingerstick reading to the blood glucose reading sensor. Based upon this comparison, the virtual patient software may instruct the blood glucose sensor to perform a calibration of 03 mg/dl in order to match the fingerstick reading. In an embodiment of the invention, the virtual patient software may display a message on the screen of the computing device to instruct the user to make the calibration. In an alternative embodiment of the invention, the virtual patient software 105 may transmit the calibration message or command directly to the blood glucose sensor 1510.

The virtual patient software 105 may also receive inputs that the patient believes would be necessary to improve the patient's therapy. In the instructive mode, these inputs are in essence test inputs to see the reaction of the patient model to the test input. For example, a basal rate proposed change may be entered into the virtual patient software 105 by a user. The virtual patient software 105 receives the adjusted inputs and/or events and enters these inputs into the patient model, which in turn calculates new values for the measured parameters based on the inputs and/or events. This updated projected information is then displayed 1616 by the virtual patient software 105 in order for the patient to see the effect of the proposed input on the selected patient model. A patient can utilize the virtual patient software 105 in order to simulate a number of these proposed changes in order to determine the interaction of these inputs on his actual measured data. The ability to simulate a number of changes is illustrated by the return arrow to step 1616. These number of proposed changes in the inputs or events may constitute a trial therapy for or by the patient. The patient may save or print this trial therapy so that it may be utilized at a later time. Note that these proposed changes in inputs or proposed events do not effect the underlying readings in the patient model or the patient model itself because these are only test inputs (or a trial therapy) and are not actual inputs that have been put into the patient. In this mode, the focus is on teaching a patient or a medical professional how certain actions affect the patient model.

If the monitoring mode has been selected for the virtual patient software, a start input time is entered 1618 for the monitoring mode. This establishes a start time or a time of the day when the patient or medical professional is interacting with the virtual patient software. Illustratively, if the patient or medical professional is monitoring the patient starting at 7:00 am, a starting time of 7:00 am is entered and the patient model provides the readings for the selected patient. This information is supplied to the graph display section 71 of the virtual patient software 105. In an embodiment of the invention, the glucose sensor 1510 has supplied the blood glucose readings for the patient (before the entered timeframe) to the virtual patient software 1550. In an embodiment of the invention, the insulin pump 1530 supplied the basal rate of the patient (before the entered timeframe) to the virtual patient software 1550. Under certain operating conditions, the patient has previously supplied other inputs such as meals and/or boluses. The virtual patient software 1550 displays whatever information has been supplied (before the entered timeframe) on the graph display section 714 of the manipulate and view menu.

In an embodiment of the invention, a user of the virtual patient software (either patient or medical professional) may enter 1620 an event or an input. The user may enter an event like a fingerstick reading and the time it was taken. If the user is utilizing a blood glucose sensor, then the fingerstick reading may be used to calibrate the blood glucose sensor, as discussed above. If the input is a meal (i.e., entered in terms of carbohydrates) and an accompanying bolus, the user enters the carbohydrate grams and the units of bolus plus the time of the input, e.g., 9:30 am.

Based on the entering of the event or input and time, the virtual patient software 1550 displays the current results at the time of the entering of the event or input. The virtual patient software 1550 displays, in the graph display section, the readings up until the entered time for the measured parameters (blood glucose level, carbohydrates consumed, insulin delivery rate). Illustratively, if 60 grams of carbohydrates are consumed with an accompanying bolus of 14.0 units, the virtual patient software displays the food input on graph 835 (the carbohydrate graph) and displays the accompanying bolus of 14.0 units on the insulin delivery graph 825. The virtual patient software also updates the readings of the blood glucose graph 840 up until the entered time or selected time. In an embodiment of the invention, the virtual patient software may receive inputs from the blood glucose sensor and may display those inputs on the blood glucose graph. In an embodiment of the invention, the virtual patient software (if no inputs are provided from a blood glucose sensor) may interpolate a current fingerstick reading and utilize an understanding of the patient's metabolic characteristics to come up with blood glucose readings for the patient in order to estimate the patient's blood glucose reading between the previous time and the entered time. This estimate is displayed on the blood glucose level graph 840 of the graph display section.

As discussed above, the virtual patient software allows a user to enter multiple events and/or inputs. This is represented by the arrow going from step 1622 to step 1620. Under certain operating conditions, the user continues to utilize the virtual patient software 1550 to monitor how the patient's therapy controls the his or her diabetes. As long as the computing device is operational, the virtual patient software 1550 continues to run. In the monitoring mode (unlike the instructive mode), the virtual patient software 1550 may store all of the readings, events, and inputs. Because the patient is utilizing the virtual patient software in a real-life environment, it is important to maintain the data. Even in embodiments of the invention where the computing device is turned off, the virtual patient software will, when it is initialized, receive readings for the timeframe that the virtual patient software was turned off or was not activated. In embodiments of the invention, the virtual patient software will prompt the user to provide inputs of information that cannot be received from, for example, the blood glucose sensor and/or the insulin pump, for the time that the virtual patient software was not activated.

The virtual patient software also allows a lab report to be generated 1626 that describes the results of the patient's therapy. As described above, the lab report shows the current performance of the patient in regard to the selected patient model with the optimization (or adjustment) that the user, e.g., the medical professional, has made. This report may be more important in the educational or instructional mode.

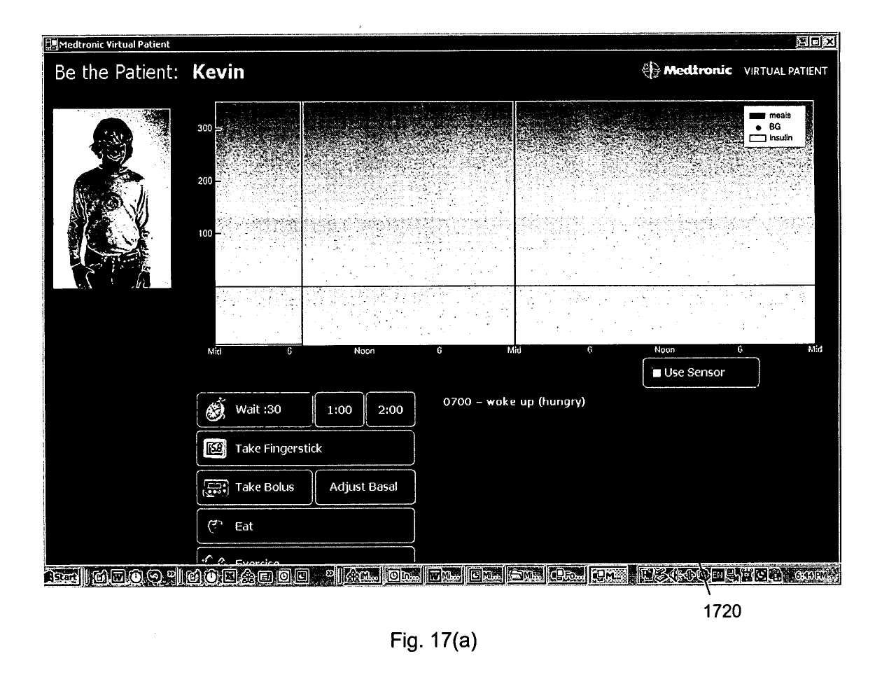

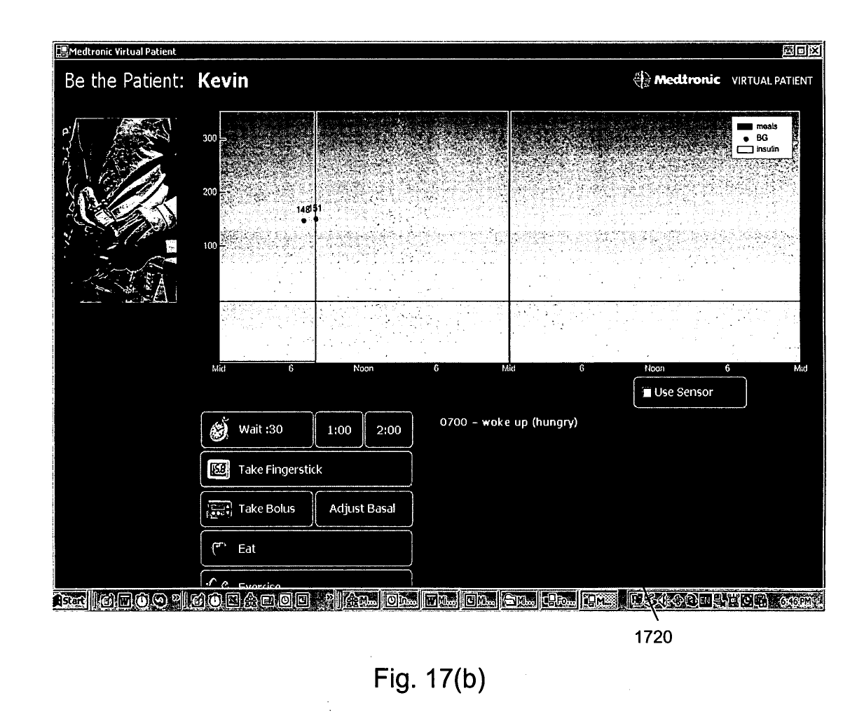

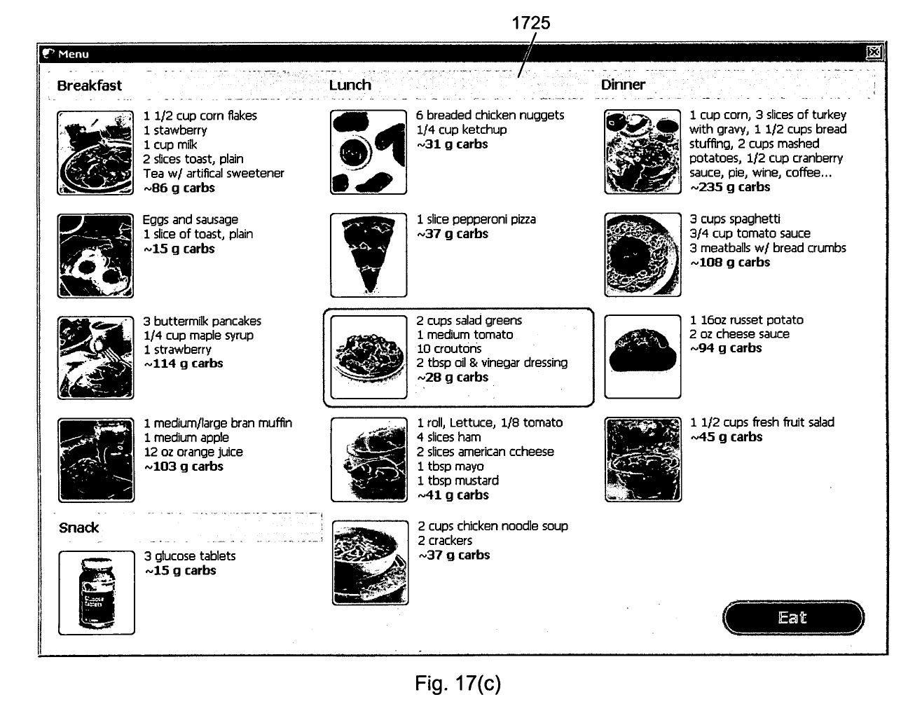

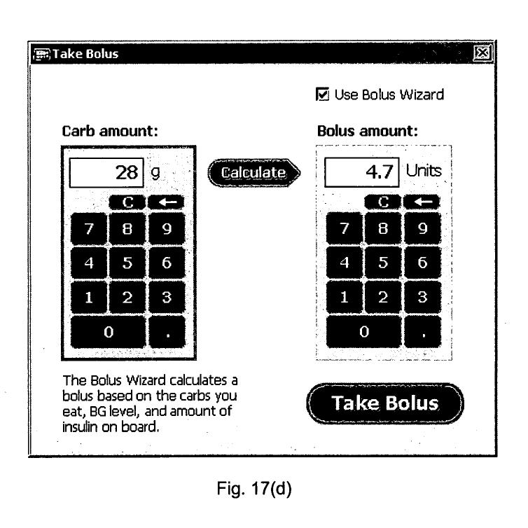

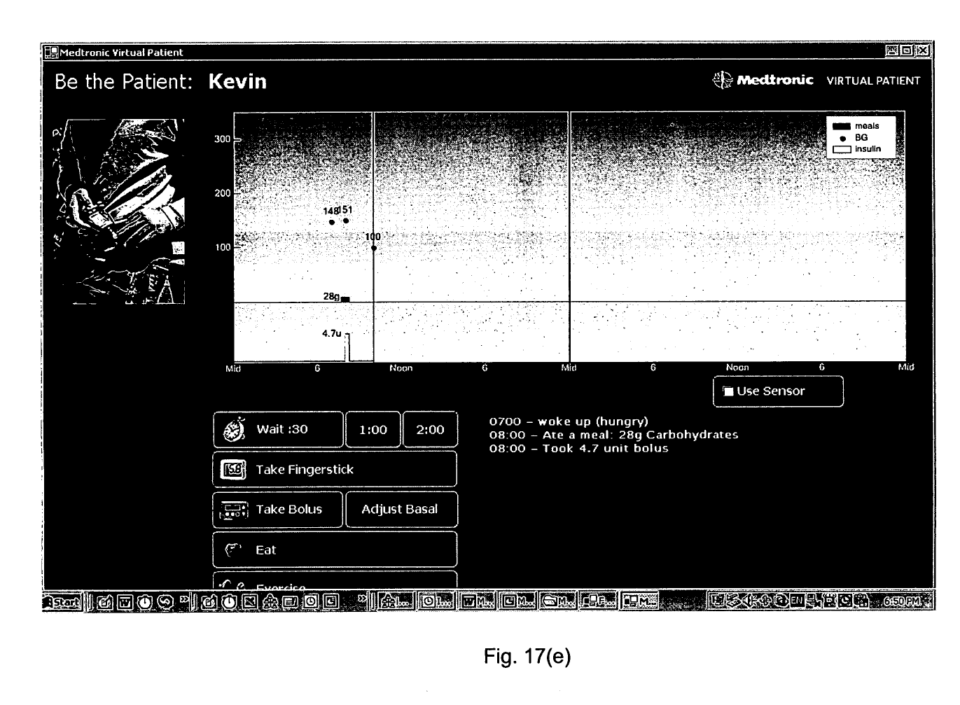

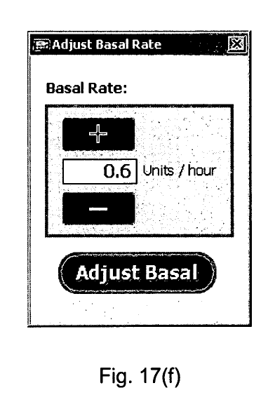

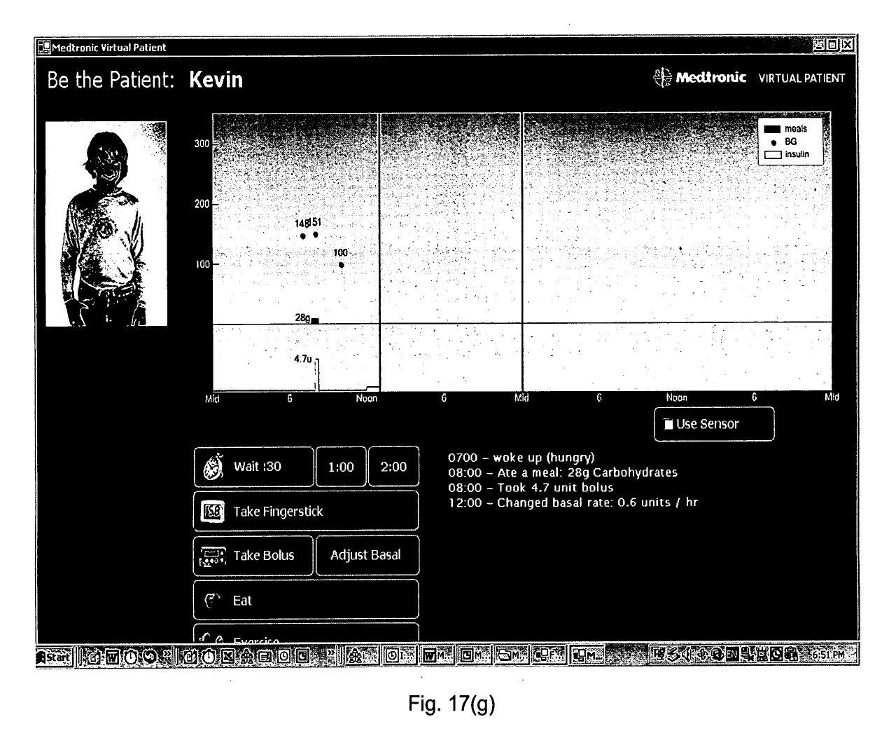

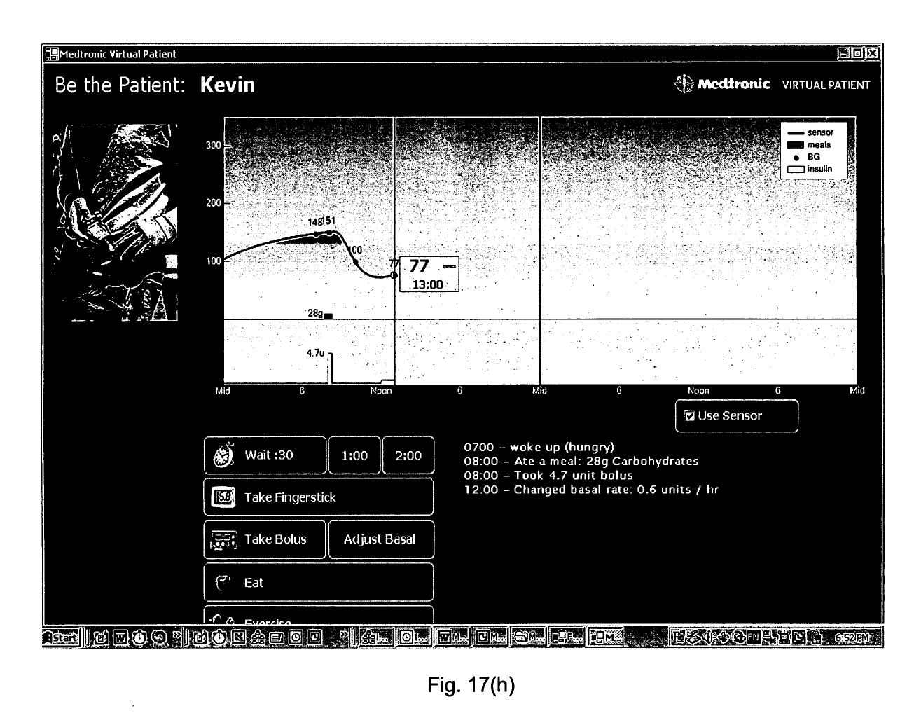

FIGS. 17(a)-17(h) illustrate a sample use of the virtual patient software in a patient mode according to an embodiment of the present invention. FIG. 17(a) illustrates a manipulate and view menu in the patient mode for the selected patient mode, e.g., Kevin. In order to arrive at FIG. 17(a) in the virtual patient software, a user selects the patient mode and selects the patient model of Kevin. A user also selects one of the time entries, e.g., 30 minutes, 1 hour, or 2 hours. After this initial selection, an activity submenu 1720 displays the time and also an event that occurred, e.g., woke up hungry. In alternative embodiments of the invention, once the patient model is selected, an initial time may automatically appear in an activity submenu 1720. The manipulate and view screen may also include an image of the selected patient.

FIG. 17(b) illustrates a manipulate and view screen in a patient mode of the virtual patient software after a plurality of fingerstick readings according to an embodiment of the present invention. In FIG. 17(b), a user has selected the “take fingerstick” selector button twice and two fingerstick readings (e.g., 148 and 151) are displayed on the blood glucose level graph 840 (see FIG. 8) of the manipulate and view screen. In an embodiment of the invention, these fingerstick readings may also be displayed in the activity submenu 1720.

FIG. 17(c) illustrates a food input screen in the virtual patient software according to an embodiment of the present invention. In order to arrive at the food input screen 1725, a user would need to select the eat toolbar or button on the manipulate and view screen of the virtual patient software. The food input screen allows a user to select a meal that exactly matches or closely approximates the meal the user has eaten or is planning to eat. A user may move a cursor over the selected meal and press enter in order to choose the selected meal. For example, in FIG. 17(c), the lunch meal of a salad (representing 28 carbohydrates (carbs) has been chosen). After the selected meal has been chosen, as illustrated by the outlined rectangle, an eat button or toolbar is selected or depressed. After the eat button or toolbar is selected, the selected meal (e.g., 28 carbohydrates) is displayed on the manipulate and view menu, specifically on a carbohydrates consumed graph 835.

After a meal is eaten, a patient generally takes a bolus to counteract the carbohydrates consumed during the meal. FIG. 17(d) illustrates a bolus wizard submenu or pop-up menu according to an embodiment of the present invention. The bolus wizard submenu converts an input number of carbohydrates into a corresponding counteracting number of bolus units. For example, as illustrated in FIG. 17(d) with the selected patient model, 28 grams of carbohydrates corresponds to about 4.7 units of insulin. After the bolus units are calculated, a take bolus button or toolbar is selected. After the take bolus button or toolbar is selected, the manipulate and view menu of the virtual patient software may display the entered bolus units on an insulin delivery graph 825, as illustrated in FIG. 17(e)

FIG. 17(e) illustrates a manipulate and view screen two hours after entering in a meal input and a bolus input according to an embodiment of the invention. A user of the virtual patient software 1550 advances the simulation two hours (by depressing one of the time entry toolbars). The user also entered a “take fingerstick” event into the system. In response, the virtual patient software 1550 manipulate and view menu displays the resulting estimated or simulated blood glucose level, e.g., 100, on the blood glucose display graph 840 the advanced to time, e.g., 10:00 am. The take bolus input, e.g., 4.7 units, is displayed on the insulin delivery graph at 8:00 am and the meal input (28 carbohydrates) is also displayed at 8:00 am on the carbohydrates consumed graph 835. The blood glucose reading displayed at 10:00 am takes into consideration both the meal input and the bolus units taken. In other words, the meal input and bolus units taken were utilized by the simulation engine 150 to calculate the 10:00 am blood glucose reading.

FIG. 17(f) illustrates a basal rate adjustment according to an embodiment of the present invention The basal rate submenu or drop-down menu allows that adjustment of the basal by tenths of units/hours. After the basal rate has been adjusted, a user selects the adjust basal button or toolbar and enters the adjusted basal rate into the virtual patient software.

FIG. 17(g) illustrates a manipulate and view screen a period of time after a basal rate has been adjusted. It is important to note that the virtual patient software 105 does not automatically or immediate modify the insulin delivery graph 825 because the basal rate has been adjusted. Under certain operating conditions, the basal rate adjustment is illustrated after the simulation has been advanced. As is illustrated in FIG. 17(g), a user enters the basal adjustment rate in at 12:00 noon and advances the simulation by one hour. The adjusted basal rate is illustrated as occurring at 12:00 noon, but is more apparent after the simulation has been advanced, in this case to 1:00 pm.

FIG. 17(h) illustrates a manipulate and view screen after a simulated blood glucose sensor feature has been enabled. In the embodiment of the invention illustrated in FIG. 17(h), a user has selected the use sensor checkbox. In response to the selection of the use sensor checkbox, the manipulate and view menu illustrates, in the blood glucose level graph 840, continuous blood glucose readings, as if they were being received from a blood glucose sensor. This is especially helpful because the user of the virtual patient software can now see a continuous set of datapoints regarding the patient's blood glucose level. The virtual patient software 1550, specifically the simulation engine 150, calculates a blood glucose reading for each minute of the simulation. This number of readings (e.g., 60 within an hour) allow the line representing the sensor readings to appear smooth. The manipulate and view menu also provides a mini pop-up window that displays the blood glucose reading at the present time of the simulation, along with other information.

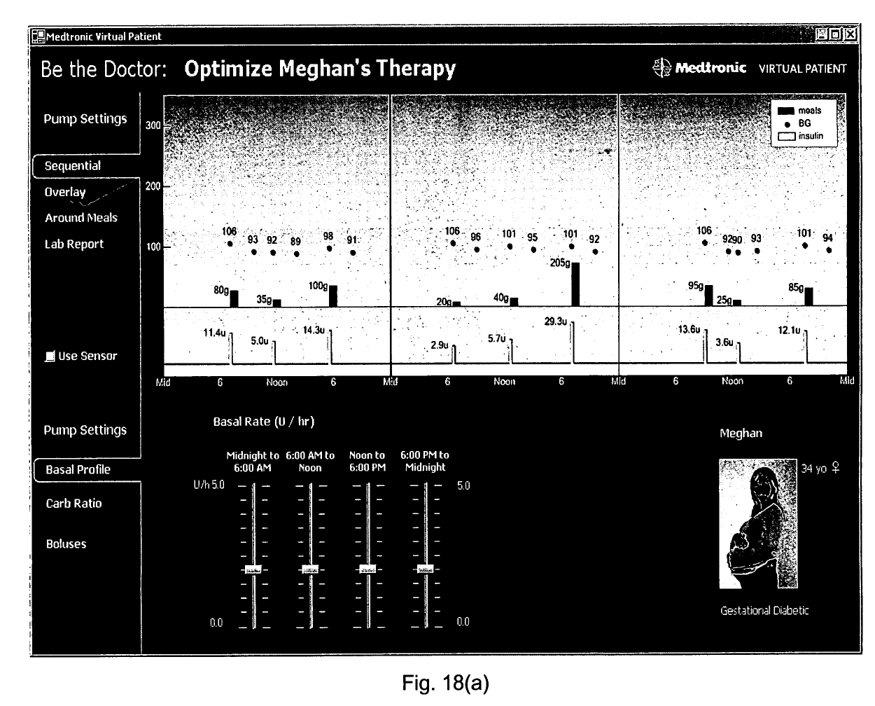

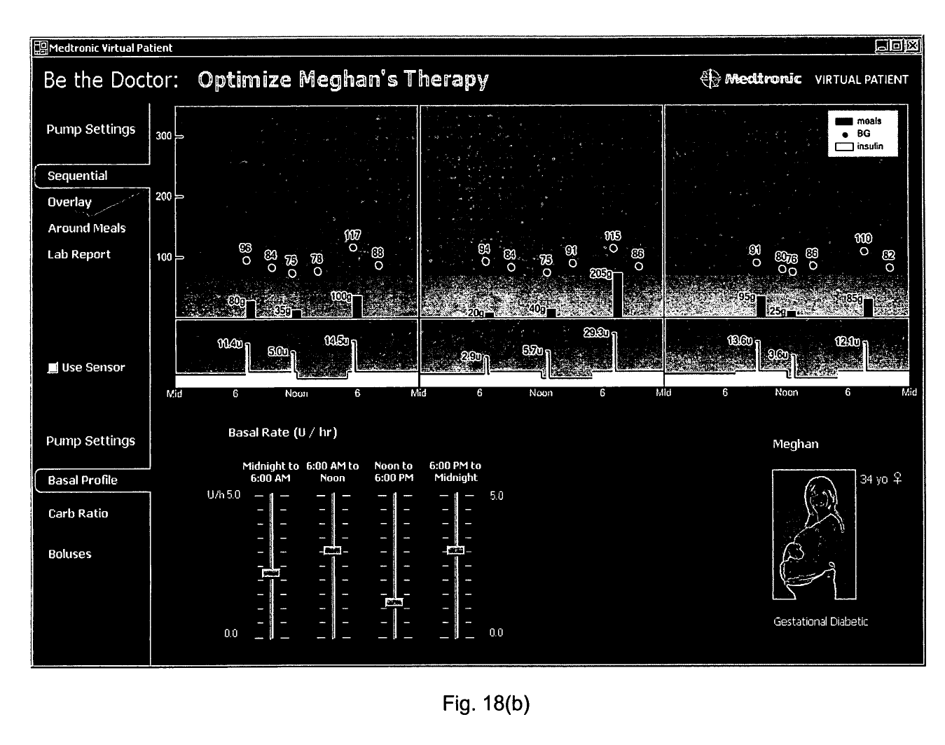

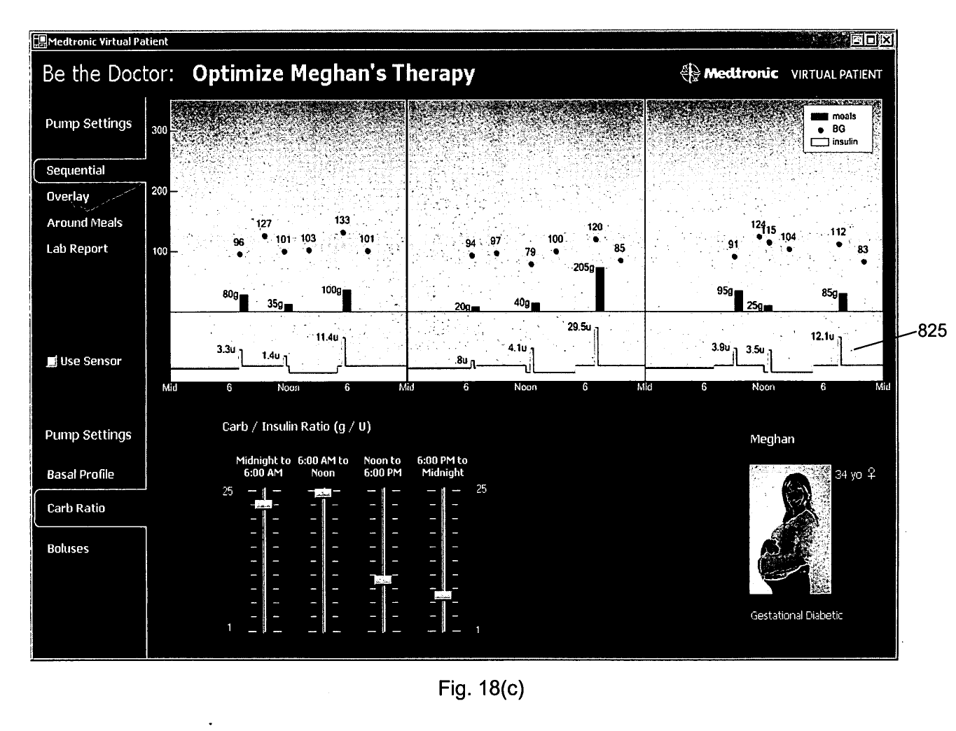

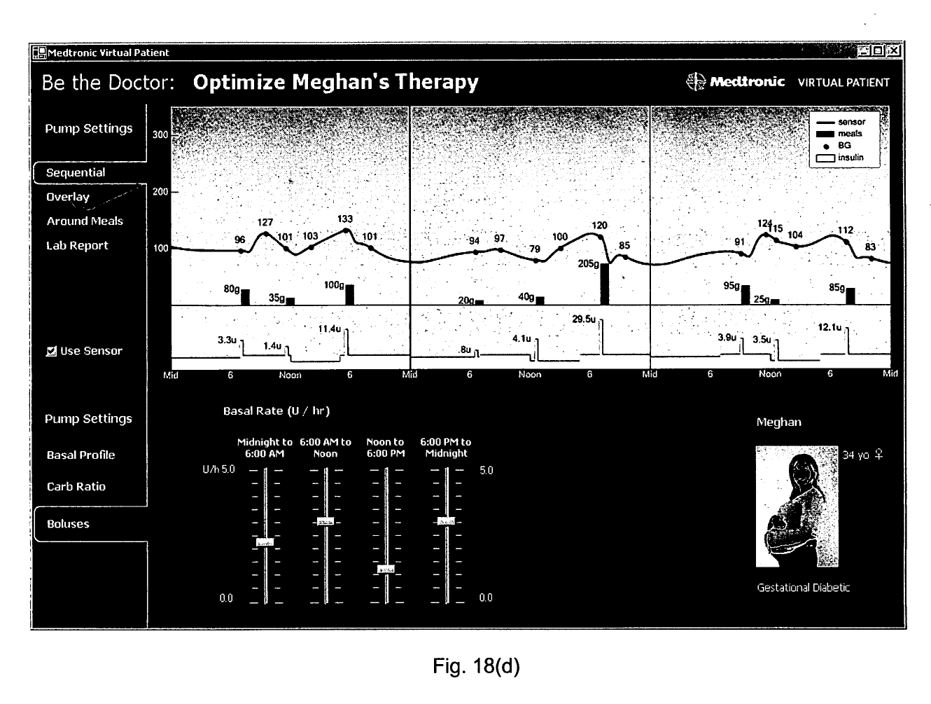

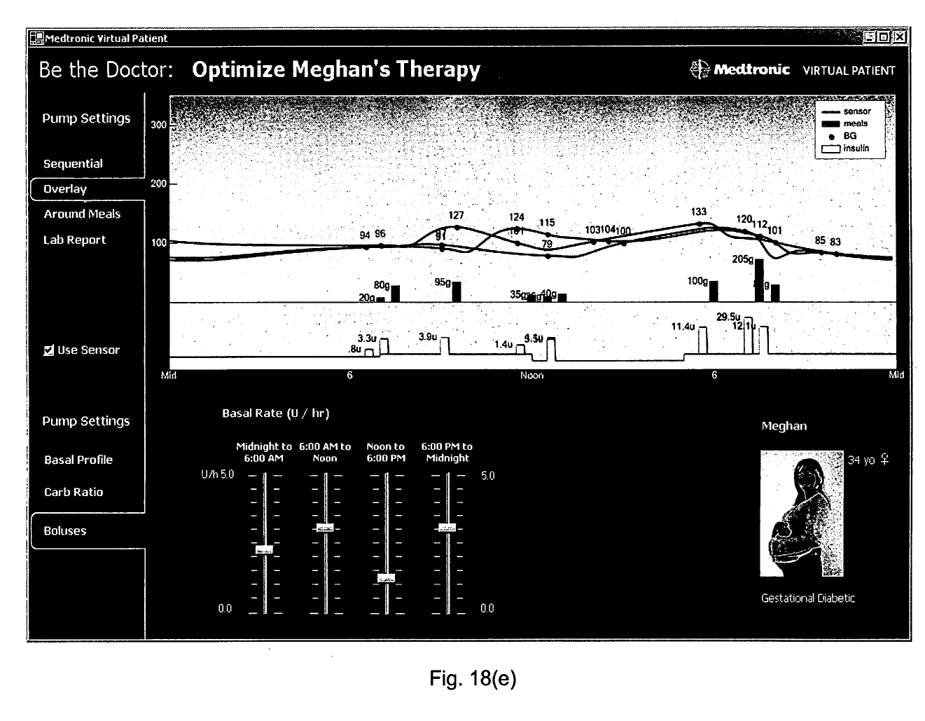

FIGS. 18(a)-18(e) illustrate a sample interaction of the doctor (or medical professional mode) according to an embodiment of the present invention. FIG. 18(a) illustrates an initial doctor mode manipulate and view screen according to an embodiment of the present invention. The doctor mode manipulate and view menu is illustrated in longitudinal fashion, i.e., having a number of days of patient data being displayed side-by-side. The doctor mode manipulate and view screen is displayed after a user selects the doctor mode and selects a patient model, e.g., Meghan in this case.

FIG. 18(b) illustrates a manipulate and view screen after an adjustment in the basal rate according to an embodiment of the present invention. As is illustrated in FIG. 18(b), the actual input toolbar has been adjusted by increasing the basal rate between 6:00 am and Noon for each of the simulation days, decreasing the basal rate between noon and 6:00 pm for each of the simulation days, and increasing the basal rate between 6:00 pm and midnight for each of the simulation days. As illustrated in FIG. 18(b), this adjustment in the basal rate produces a change in the blood glucose level of the simulated patient. For example, the second datapoint on the blood glucose level graph (which represents the blood glucose level at around 9:00 am) in each of the simulated days is significantly decreased The fifth datapoint on the blood glucose level graph (which represents a blood glucose reading at 6:00 pm) in each of the simulated days has been increased, as compared to the pre-adjustment readings.

FIG. 18(c) illustrates an adjustment in a carb/insulin ratio and a resulting graph on a manipulate and view screen of the virtual patient software according to an embodiment of the present invention. Originally, the sliders for the carb/insulin ratio were placed under the fourth indicator (e.g., where the noon to six p.m. slider is placed in FIG. 18(c)). As illustrated in FIG. 18(c), the carb/insulin ratio has been increased during both the midnight to 6:00 am timeframe and the 6:00 am to 12 noon timeframe. The carb/insulin ratio has been decreased during the 6:00 pm to midnight timeframe. The resulting impact in the blood glucose level is illustrated in FIG. 18(c). It should also be noted that the datapoints on the insulin delivery graph 825 are recalculated when modifications are made to the carb/insulin rations for different timeframes. Illustratively, the insulin delivered, as shown by FIG. 18(c) has been significantly decreased for the timeframes where the carb/insulin ration has been increased.

FIG. 18(d) illustrates a manipulate and view screen of the doctor mode of the virtual patient software including an active use sensor checkbox according to an embodiment of the present invention. After a use sensor checkbox is selected, the blood glucose level graph of the manipulate and view menu displays simulated continuous blood glucose readings for the patient. The continuous blood glucose readings provides the doctor with a better estimation of the patient's actual blood glucose behavior because it provides a display of readings and trends between the previously displayed datapoints. In an embodiment of the invention, the simulation engine generates a blood glucose reading for every minute of the simulation

FIG. 18(e) illustrates a modal viewing mode of a manipulate and view screen according to an embodiment of the present invention. A modal viewing mode allows a user to see a number of days of data superimposed on each other. This may be helpful in attempting to establish trends in a patient and to identify if there are any periods during the day or evening where the patient has a hard time maintaining the desired blood glucose level. Each of the lines in the blood glucose level graph 840 represents a simulated day. Under most operating conditions, the graphed time period in the modal viewing mode is one 24 hour day.

While the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

What is claimed is:

1. A system to assist an individual in developing a therapy for diabetes treatment of a patient, comprising:
a user interface control module to receive an input related to the patient and receive a current time of a simulation;
a simulation engine to receive the input, to generate a plurality of blood glucose readings for the patient up to the current time of the simulation based on the input, and to transfer the plurality of blood glucose readings; and
a charting and display module to receive the plurality of blood glucose readings and to display the plurality of blood glucose readings.

2. The system of claim 1, wherein the simulation engine receives patient parameters from a patient parameter library based on a selected patient model.

3. The system of claim 2, further including a stored scenarios library to store a plurality of scenarios, each of the scenarios representing a number of readings for the selected patient model, wherein one of the plurality of scenarios is selected along with the selection of the patient model.

4. The system of claim 1, further including a food data library housing a plurality of meal inputs, wherein the input is selected from one of the plurality of meal inputs.

5. The system of claim 4, wherein the user interface control module transfers the meal input to the charting and display module and the charting and display module displays a representation of the meal input

6. The system of claim 5, wherein the meal input is entered as a number of carbohydrates.

7. A system to assist an individual in developing a therapy for diabetes treatment of a patient utilizing actual data of the patient, comprising:
a user interface control module to receive an adjustment to an input, the input relating to the therapy;
a simulation engine to receive the adjustment to the input, to generate a plurality of blood glucose readings for the patient over a simulation timeframe based on the adjustment of the input, and to transfer the plurality of blood glucose readings; and
a charting and display module to receive the plurality of blood glucose readings and to modify a display of currently presented blood glucose readings based on the received plurality of blood glucose readings.

8. The system of claim 7, further including a storage to store actual patient data, wherein the actual patient data is transferred to the user interface control module.

9. The system of claim 8, wherein the actual patient data is transferred from the user interface control module to the charting and display module and the charting and display module displays at least a portion of the input actual patient data on a display of the computing device.

10. The system of claim 7, further including a storage to store actual patient data and a patient parameter fit module to determine mathematical parameters for the simulation engine, wherein the input actual patient data is transferred to the patient parameter fit module in order for the patient parameter fit module to determine the mathematical parameters.

11. The system of claim 10, wherein the simulation engine uses the mathematical parameters along with the adjustment to the input to create the plurality of blood glucose readings.

12. The system of claim 8, wherein the actual patient data is transmitted from at least one of a blood glucose sensor and an insulin pump to the storage in the system

13. The system of claim 12, wherein the actual patient data is transmitted utilizing wireless communications.

14. A method to assist an individual in developing a therapy to aid diabetes management for a patient by running a simulation, comprising:
receiving an input or an event related to the diabetes management of the patient in a patient mode and capturing a first time of the simulation;
calculating a plurality of simulated blood glucose readings, up to the first time of the simulation, for the patient based on the received input or event; and
displaying the plurality of simulated blood glucose readings for the patient.

15. The method of claim 14, where the displaying of the plurality of simulated blood glucose readings occurs in a time proximate to and after the receiving of the input or the event.

16. The method of claim 14, further including providing on-screen assistance after selecting a patient model of operation.

17. The method of claim 14, further including generating and displaying a lab report to detail the individual's success in developing the therapy for diabetes management.

18. The method of claim 14, further including displaying a representation of the first input in an area of a display separate from the display of the plurality of simulated blood glucose readings.

19. The method of claim 14, further including receiving a second input or event and capturing a second time of the simulation;
calculating a second plurality of simulated blood glucose readings for the patient based on the received second input and event for the timeframe between the first time of the simulation and the second time of the simulation; and
displaying the second plurality of simulated blood glucose readings for the timeframe between the first time of the simulation and the second time of the simulation.

20. A program code storage device, comprising:
a computer-readable storage media; and
computer-readable program code, stored on the computer-readable media, the computer-readable program code including instructions, which when executed cause a computing device to:
receive an input or an event related to the diabetes management of the patient in a patient mode and capture a first time of a simulation;
calculate a plurality simulated blood glucose readings, up to the first time of the simulation, for the patient based on the received input or event; and
display the plurality of simulated blood glucose readings for the patient.

21. The program code storage device of claim 20, wherein the displaying of the plurality of simulated blood glucose readings occurs in a time proximate to and after receiving of the input or the event.

22. The computer program code storage device of claim 20, the computer-readable program code including instructions, which when executed cause the computing device to provide on-screen assistance after selecting the patient model of operation.

23. The computer program code storage device of claim 20, the computer-readable program code including instructions, which when executed, cause the computing device to generate and display a lab report to detail the individual's success in developing the therapy for diabetes management.

24. The computer program code storage device of claim 20, the computer-readable program code including instructions, which when executed, cause the computing device to display a representation of the first input in an area of a display separate from the display of the plurality of simulated blood glucose readings.

25. The computer program code storage device of claim 20, the computer-readable program code including instructions, which when executed, cause the computing device to:
receive a second input or event and capturing a second time of the simulation,
calculate a second plurality of simulated blood glucose readings for the patient based on the received second input or event between the first time of the simulation and the second time of the simulation; and
display the second plurality of simulated blood glucose readings for the patient for the timeframe between the first time of the simulation up until the second time of the simulation.

26. A method to assist a doctor in developing a therapy for diabetes management of a patient by running a simulation, comprising:
receiving an input related to the diabetes management of the patient in a doctor mode;
calculating a plurality of simulated blood glucose readings for the timeframe of the simulation, for the patient based on the received input; and
displaying the plurality of simulated blood glucose readings for the timeframe of the simulation on a display.

27. The method of claim 26, wherein the displaying of the plurality of simulated blood glucose readings occurs in a time proximate to and after the receiving of the input.

28. The method of claim 26, further including receiving a second input;
calculating a second plurality of simulated blood glucose readings for the patient based on the received second input for the timeframe of the simulation; and
displaying the second plurality of simulated blood glucose readings on the display.

29. A program code storage device, comprising:
a computer-readable storage media; and
computer-readable program code, stored on the computer-readable storage media, the computer-readable program code including instructions, which when executed cause a computing device to:
receive an input related to the diabetes management of the patient in a doctor mode;
calculating a plurality of simulated blood glucose readings for a timeframe of a simulation, for the patient based on the received input; and
displaying the plurality of simulated blood glucose readings for the timeframe of the simulation on a display.

30. The program code storage device of claim 29, wherein the displaying of the plurality of simulated blood glucose readings occurs in a time proximate to and after the receiving of the input.

31. The program code storage device of claim 29, the computer-readable program code including instructions, which when executed cause the computing device to:
receive a second input;
calculate a second plurality of simulated blood glucose readings for the patient based on the received second input for the timeframe of the simulation; and
display the second plurality of simulated blood glucose readings on the display.
